SEQUENCE LISTING (32 sequences)

SEQ ID NO: 1 - RNA, 22 nt, synthetic construct
  source 1..22: mol_type = other RNA
  modified_base 1: mod_base = OTHER; note = 2'-a GalNAc clicked cytidine
  modified_base 1..3: mod_base = OTHER; note = Residues are connected through a phosphorothioate linkage
  modified_base 2: mod_base = OTHER; note = Residue has a 2'-deoxy-2'-fluoro (2'-F) ribosugar modification
  modified_base 4: mod_base = OTHER; note = Residue has a 2'-deoxy-2'-fluoro (2'-F) ribosugar modification
  modified_base 3: mod_base = gm
  modified_base 5: mod_base = gm
  modified_base 6: mod_base = OTHER; note = Residue has a 2'-deoxy-2'-fluoro (2'-F) ribosugar modification
  modified_base 7: mod_base = gm
  modified_base 8: mod_base = OTHER; note = Residue has a 2'-deoxy-2'-fluoro (2'-F) ribosugar modification
  modified_base 9: mod_base = gm
  modified_base 10: mod_base = OTHER; note = Residue has a 2'-deoxy-2'-fluoro (2'-F) ribosugar modification
  modified_base 11: mod_base = gm
  modified_base 12: mod_base = OTHER; note = Residue has a 2'-deoxy-2'-fluoro (2'-F) ribosugar modification
  modified_base 13: mod_base = um
  modified_base 14: mod_base = OTHER; note = Residue has a 2'-deoxy-2'-fluoro (2'-F) ribosugar modification
  modified_base 15: mod_base = um
  modified_base 16: mod_base = OTHER; note = Residue has a 2'-deoxy-2'-fluoro (2'-F) ribosugar modification
  modified_base 17: mod_base = um
  modified_base 18: mod_base = OTHER; note = Residue has a 2'-deoxy-2'-fluoro (2'-F) ribosugar modification
  modified_base 19: mod_base = cm
  modified_base 20: mod_base = OTHER; note = Residue has a 2'-deoxy-2'-fluoro (2'-F) ribosugar modification
  modified_base 21: mod_base = cm
  modified_base 22: mod_base = OTHER; note = Residue has a 2'-deoxy-2'-fluoro (2'-F) ribosugar modification
  modified_base 21^22: mod_base = OTHER; note = Residues are connected through a phosphorothioate linkage
ctgggagagggttgtttactcc

SEQ ID NO: 2 - RNA, 22 nt, synthetic construct
  source 1..22: mol_type = other RNA
  modified_base 1^2: mod_base = OTHER; note = Residues are connected through a phosphorothioate linkage
  modified_base 19..22: mod_base = OTHER; note = Residues are connected through a phosphorothioate linkage
  modified_base 1: mod_base = OTHER; note = 2'-a GalNAc clicked cytidine
  modified_base 2: mod_base = OTHER; note = Residue has a 2'-deoxy-2'-fluoro (2'-F) ribosugar modification
  modified_base 3: mod_base = gm
  modified_base 4: mod_base = OTHER; note = Residue has a 2'-deoxy-2'-fluoro (2'-F) ribosugar modification
  modified_base 5: mod_base = gm
  modified_base 6: mod_base = OTHER; note = Residue has a 2'-deoxy-2'-fluoro (2'-F) ribosugar modification
  modified_base 7: mod_base = gm
  modified_base 8: mod_base = OTHER; note = Residue has a 2'-deoxy-2'-fluoro (2'-F) ribosugar modification
  modified_base 9: mod_base = gm
  modified_base 10: mod_base = OTHER; note = Residue has a 2'-deoxy-2'-fluoro (2'-F) ribosugar modification
  modified_base 11: mod_base = gm
  modified_base 12: mod_base = OTHER; note = Residue has a 2'-deoxy-2'-fluoro (2'-F) ribosugar modification
  modified_base 13: mod_base = um
  modified_base 14: mod_base = OTHER; note = Residue has a 2'-deoxy-2'-fluoro (2'-F) ribosugar modification
  modified_base 15: mod_base = um
  modified_base 16: mod_base = OTHER; note = Residue has a 2'-deoxy-2'-fluoro (2'-F) ribosugar modification
  modified_base 17: mod_base = um
  modified_base 18: mod_base = OTHER; note = Residue has a 2'-deoxy-2'-fluoro (2'-F) ribosugar modification
  modified_base 19: mod_base = cm
  modified_base 20: mod_base = OTHER; note = Residue has a 2'-deoxy-2'-fluoro (2'-F) ribosugar modification
  modified_base 21: mod_base = cm
  modified_base 22: mod_base = OTHER; note = Residue has a 2'-deoxy-2'-fluoro (2'-F) ribosugar modification
ctgggagagggttgtttactcc

SEQ ID NO: 3 - RNA, 22 nt, synthetic construct
  source 1..22: mol_type = other RNA
  modified_base 1..3: mod_base = OTHER; note = Residues are connected through a phosphorothioate linkage
  modified_base 19..22: mod_base = OTHER; note = Residues are connected through a phosphorothioate linkage
  modified_base 1: mod_base = OTHER; note = 2'-a GalNAc clicked cytidine
  modified_base 2: mod_base = OTHER; note = Residue has a 2'-deoxy-2'-fluoro (2'-F) ribosugar modification
  modified_base 3: mod_base = gm
  modified_base 4: mod_base = OTHER; note = Residue has a 2'-deoxy-2'-fluoro (2'-F) ribosugar modification
  modified_base 5: mod_base = gm
  modified_base 6: mod_base = OTHER; note = Residue has a 2'-deoxy-2'-fluoro (2'-F) ribosugar modification
  modified_base 7: mod_base = gm
  modified_base 8: mod_base = OTHER; note = Residue has a 2'-deoxy-2'-fluoro (2'-F) ribosugar modification
  modified_base 9: mod_base = gm
  modified_base 10: mod_base = OTHER; note = Residue has a 2'-deoxy-2'-fluoro (2'-F) ribosugar modification
  modified_base 11: mod_base = gm
  modified_base 12: mod_base = OTHER; note = Residue has a 2'-deoxy-2'-fluoro (2'-F) ribosugar modification
  modified_base 13: mod_base = um
  modified_base 14: mod_base = OTHER; note = Residue has a 2'-deoxy-2'-fluoro (2'-F) ribosugar modification
  modified_base 15: mod_base = um
  modified_base 16: mod_base = OTHER; note = Residue has a 2'-deoxy-2'-fluoro (2'-F) ribosugar modification
  modified_base 17: mod_base = um
  modified_base 18: mod_base = OTHER; note = Residue has a 2'-deoxy-2'-fluoro (2'-F) ribosugar modification
  modified_base 19: mod_base = cm
  modified_base 20: mod_base = OTHER; note = Residue has a 2'-deoxy-2'-fluoro (2'-F) ribosugar modification
  modified_base 21: mod_base = cm
  modified_base 22: mod_base = OTHER; note = Residue has a 2'-deoxy-2'-fluoro (2'-F) ribosugar modification
ctgggagagggttgtttactcc

SEQ ID NO: 4 - RNA, 21 nt, synthetic construct
  source 1..21: mol_type = other RNA
  modified_base 20^21: mod_base = OTHER; note = Residues are connected through a phosphorothioate linkage
  modified_base 1: mod_base = OTHER; note = Residue has a 2'-deoxy-2'-fluoro (2'-F) ribosugar modification
  modified_base 2: mod_base = gm
  modified_base 3: mod_base = OTHER; note = Residue has a 2'-deoxy-2'-fluoro (2'-F) ribosugar modification
  modified_base 4: mod_base = OTHER; note = Residue is 2'-O-methylated
  modified_base 5: mod_base = OTHER; note = Residue has a 2'-deoxy-2'-fluoro (2'-F) ribosugar modification
  modified_base 6: mod_base = OTHER; note = Residue is 2'-O-methylated
  modified_base 7: mod_base = OTHER; note = Residue has a 2'-deoxy-2'-fluoro (2'-F) ribosugar modification
  modified_base 8: mod_base = gm
  modified_base 9: mod_base = OTHER; note = Residue has a 2'-deoxy-2'-fluoro (2'-F) ribosugar modification
  modified_base 10: mod_base = um
  modified_base 11: mod_base = OTHER; note = Residue has a 2'-deoxy-2'-fluoro (2'-F) ribosugar modification
  modified_base 12: mod_base = gm
  modified_base 13: mod_base = OTHER; note = Residue has a 2'-deoxy-2'-fluoro (2'-F) ribosugar modification
  modified_base 14: mod_base = um
  modified_base 15: mod_base = OTHER; note = Residue has a 2'-deoxy-2'-fluoro (2'-F) ribosugar modification
  modified_base 16: mod_base = OTHER; note = Residue is 2'-O-methylated
  modified_base 17: mod_base = OTHER; note = Residue has a 2'-deoxy-2'-fluoro (2'-F) ribosugar modification
  modified_base 18: mod_base = um
  modified_base 19: mod_base = OTHER; note = Residue has a 2'-deoxy-2'-fluoro (2'-F) ribosugar modification
  modified_base 20: mod_base = OTHER; note = 2'-a GalNAc clicked cytidine
  modified_base 21: mod_base = um
gggagagggttgtttactcct

SEQ ID NO: 5 - RNA, 21 nt, synthetic construct
  source 1..21: mol_type = other RNA
  modified_base 18..21: mod_base = OTHER; note = Residues are connected through a phosphorothioate linkage
  modified_base 1: mod_base = OTHER; note = Residue has a 2'-deoxy-2'-fluoro (2'-F) ribosugar modification
  modified_base 2: mod_base = gm
  modified_base 3: mod_base = OTHER; note = Residue has a 2'-deoxy-2'-fluoro (2'-F) ribosugar modification
  modified_base 4: mod_base = OTHER; note = Residue is 2'-O-methylated
  modified_base 5: mod_base = OTHER; note = Residue has a 2'-deoxy-2'-fluoro (2'-F) ribosugar modification
  modified_base 6: mod_base = OTHER; note = Residue is 2'-O-methylated
  modified_base 7: mod_base = OTHER; note = Residue has a 2'-deoxy-2'-fluoro (2'-F) ribosugar modification
  modified_base 8: mod_base = gm
  modified_base 9: mod_base = OTHER; note = Residue has a 2'-deoxy-2'-fluoro (2'-F) ribosugar modification
  modified_base 10: mod_base = um
  modified_base 11: mod_base = OTHER; note = Residue has a 2'-deoxy-2'-fluoro (2'-F) ribosugar modification
  modified_base 12: mod_base = gm
  modified_base 13: mod_base = OTHER; note = Residue has a 2'-deoxy-2'-fluoro (2'-F) ribosugar modification
  modified_base 14: mod_base = um
  modified_base 15: mod_base = OTHER; note = Residue has a 2'-deoxy-2'-fluoro (2'-F) ribosugar modification
  modified_base 16: mod_base = OTHER; note = Residue is 2'-O-methylated
  modified_base 17: mod_base = OTHER; note = Residue has a 2'-deoxy-2'-fluoro (2'-F) ribosugar modification
  modified_base 18: mod_base = um
  modified_base 19: mod_base = OTHER; note = Residue has a 2'-deoxy-2'-fluoro (2'-F) ribosugar modification
  modified_base 20: mod_base = OTHER; note = 2'-a GalNAc clicked cytidine
  modified_base 21: mod_base = um
gggagagggttgtttactcct

SEQ ID NO: 6 - RNA, 23 nt, synthetic construct
  source 1..23: mol_type = other RNA
  modified_base 1..3: mod_base = OTHER; note = Residues are connected through a phosphorothioate linkage
  modified_base 1: mod_base = OTHER; note = Residue has a 2'-deoxy-2'-fluoro (2'-F) ribosugar modification
  modified_base 2: mod_base = um
  modified_base 3: mod_base = OTHER; note = Residue has a 2'-deoxy-2'-fluoro (2'-F) ribosugar modification
  modified_base 4: mod_base = gm
  modified_base 5: mod_base = OTHER; note = Residue has a 2'-deoxy-2'-fluoro (2'-F) ribosugar modification
  modified_base 6: mod_base = OTHER; note = Residue is 2'-O-methylated
  modified_base 7: mod_base = OTHER; note = Residue has a 2'-deoxy-2'-fluoro (2'-F) ribosugar modification
  modified_base 8: mod_base = OTHER; note = Residue is 2'-O-methylated
  modified_base 9: mod_base = OTHER; note = Residue has a 2'-deoxy-2'-fluoro (2'-F) ribosugar modification
  modified_base 10: mod_base = gm
  modified_base 11: mod_base = OTHER; note = Residue has a 2'-deoxy-2'-fluoro (2'-F) ribosugar modification
  modified_base 12: mod_base = um
  modified_base 13: mod_base = OTHER; note = Residue has a 2'-deoxy-2'-fluoro (2'-F) ribosugar modification
  modified_base 14: mod_base = gm
  modified_base 15: mod_base = OTHER; note = Residue has a 2'-deoxy-2'-fluoro (2'-F) ribosugar modification
  modified_base 16: mod_base = um
  modified_base 17: mod_base = OTHER; note = Residue has a 2'-deoxy-2'-fluoro (2'-F) ribosugar modification
  modified_base 18: mod_base = OTHER; note = Residue is 2'-O-methylated
  modified_base 19: mod_base = OTHER; note = Residue has a 2'-deoxy-2'-fluoro (2'-F) ribosugar modification
  modified_base 20: mod_base = um
  modified_base 21: mod_base = OTHER; note = Residue has a 2'-deoxy-2'-fluoro (2'-F) ribosugar modification
  modified_base 22: mod_base = OTHER; note = 2'-a GalNAc clicked cytidine
  modified_base 23: mod_base = um
ctgggagagggttgtttactcct

SEQ ID NO: 7 - RNA, 23 nt, synthetic construct
  source 1..23: mol_type = other RNA
tgtaaacatcctacactctcagc

SEQ ID NO: 8 - RNA, 22 nt, synthetic construct
  source 1..22: mol_type = other RNA
ctgggagagggttgtttactcc

SEQ ID NO: 9 - RNA, 22 nt, synthetic construct
  source 1..22: mol_type = other RNA
ctgggagaaggctgtttactct

SEQ ID NO: 10 - DNA, 21 bp, synthetic construct
  source 1..21: mol_type = other DNA
aaggagcgtaggtctttgcag

SEQ ID NO: 11 - DNA, 20 bp, synthetic construct
  source 1..20: mol_type = other DNA
agagctacgagctgcctgac

SEQ ID NO: 12 - DNA, 20 bp, synthetic construct
  source 1..20: mol_type = other DNA
agcactgtgttggcgtacag

SEQ ID NO: 13 - RNA, 24 nt, synthetic construct
  source 1..24: mol_type = other RNA
tcacaacctcctagaaagagtaga

SEQ ID NO: 14 - RNA, 23 nt, synthetic construct
  source 1..23: mol_type = other RNA
tgtaaacatcctacactctcagc

SEQ ID NO: 15 - RNA, 22 nt, synthetic construct
  source 1..22: mol_type = other RNA
  modified_base 2: mod_base = um
  modified_base 3: mod_base = gm
  modified_base 4: mod_base = gm
  modified_base 5: mod_base = gm
  modified_base 6: mod_base = OTHER; note = Residue is 2'-O-methylated
  modified_base 7: mod_base = gm
  modified_base 8: mod_base = OTHER; note = Residue is 2'-O-methylated
  modified_base 9: mod_base = gm
  modified_base 10: mod_base = gm
  modified_base 11: mod_base = gm
  modified_base 12: mod_base = um
  modified_base 13: mod_base = um
  modified_base 14: mod_base = gm
  modified_base 15: mod_base = um
  modified_base 16: mod_base = um
  modified_base 17: mod_base = um
  modified_base 18: mod_base = OTHER; note = Residue is 2'-O-methylated
  modified_base 19: mod_base = cm
  modified_base 20: mod_base = um
  modified_base 21: mod_base = cm
ctgggagagggttgtttactcc

SEQ ID NO: 16 - RNA, 22 nt, synthetic construct
  source 1..22: mol_type = other RNA
  modified_base 2: mod_base = p
  modified_base 3: mod_base = gm
  modified_base 4: mod_base = gm
  modified_base 5: mod_base = gm
  modified_base 6: mod_base = OTHER; note = Residue is 2'-O-methylated
  modified_base 7: mod_base = gm
  modified_base 8: mod_base = OTHER; note = Residue is 2'-O-methylated
  modified_base 9: mod_base = gm
  modified_base 10: mod_base = gm
  modified_base 11: mod_base = gm
  modified_base 12: mod_base = p
  modified_base 13: mod_base = p
  modified_base 14: mod_base = gm
  modified_base 15: mod_base = p
  modified_base 16: mod_base = p
  modified_base 17: mod_base = p
  modified_base 18: mod_base = OTHER; note = Residue is 2'-O-methylated
  modified_base 19: mod_base = cm
  modified_base 20: mod_base = p
  modified_base 21: mod_base = cm
cngggagagggnngnnnacncc

SEQ ID NO: 17 - RNA, 22 nt, synthetic construct
  source 1..22: mol_type = other RNA
  modified_base 2: mod_base = p
  modified_base 3: mod_base = gm
  modified_base 4: mod_base = gm
  modified_base 5: mod_base = gm
  modified_base 7: mod_base = gm
  modified_base 9: mod_base = gm
  modified_base 10: mod_base = gm
  modified_base 11: mod_base = gm
  modified_base 12: mod_base = p
  modified_base 13: mod_base = p
  modified_base 14: mod_base = gm
  modified_base 15: mod_base = p
  modified_base 16: mod_base = p
  modified_base 17: mod_base = p
  modified_base 20: mod_base = p
cngggagagggnngnnnacncc

SEQ ID NO: 18 - RNA, 22 nt, synthetic construct
  source 1..22: mol_type = other RNA
  modified_base 2: mod_base = um
  modified_base 3: mod_base = gm
  modified_base 4: mod_base = gm
  modified_base 5: mod_base = gm
  modified_base 6: mod_base = OTHER; note = Residue is 2'-O-methylated
  modified_base 7: mod_base = gm
  modified_base 8: mod_base = OTHER; note = Residue is 2'-O-methylated
  modified_base 9: mod_base = OTHER; note = Residue is 2'-O-methylated
  modified_base 10: mod_base = gm
  modified_base 11: mod_base = gm
  modified_base 12: mod_base = cm
  modified_base 13: mod_base = um
  modified_base 14: mod_base = gm
  modified_base 15: mod_base = um
  modified_base 16: mod_base = um
  modified_base 17: mod_base = um
  modified_base 18: mod_base = OTHER; note = Residue is 2'-O-methylated
  modified_base 19: mod_base = cm
  modified_base 20: mod_base = um
  modified_base 21: mod_base = cm
ctgggagaaggctgtttactct

SEQ ID NO: 19 - RNA, 22 nt, synthetic construct
  source 1..22: mol_type = other RNA
  modified_base 1: mod_base = cm
  modified_base 2: mod_base = p
  modified_base 3: mod_base = gm
  modified_base 4: mod_base = gm
  modified_base 5: mod_base = gm
  modified_base 6: mod_base = OTHER; note = Residue is 2'-O-methylated
  modified_base 7: mod_base = gm
  modified_base 8: mod_base = OTHER; note = Residue is 2'-O-methylated
  modified_base 9: mod_base = OTHER; note = Residue is 2'-O-methylated
  modified_base 10: mod_base = gm
  modified_base 11: mod_base = gm
  modified_base 12: mod_base = cm
  modified_base 13: mod_base = p
  modified_base 14: mod_base = gm
  modified_base 15: mod_base = p
  modified_base 16: mod_base = p
  modified_base 17: mod_base = p
  modified_base 18: mod_base = OTHER; note = Residue is 2'-O-methylated
  modified_base 19: mod_base = cm
  modified_base 20: mod_base = p
  modified_base 21: mod_base = cm
cngggagaaggcngnnnacnct

SEQ ID NO: 20 - RNA, 22 nt, synthetic construct
  source 1..22: mol_type = other RNA
  modified_base 2: mod_base = p
  modified_base 3: mod_base = gm
  modified_base 4: mod_base = gm
  modified_base 7: mod_base = gm
  modified_base 10: mod_base = gm
  modified_base 13: mod_base = p
  modified_base 11: mod_base = gm
  modified_base 14: mod_base = gm
  modified_base 15: mod_base = p
  modified_base 16: mod_base = p
  modified_base 17: mod_base = p
  modified_base 20: mod_base = p
  modified_base 5: mod_base = gm
cngggagaaggcngnnnacnct

SEQ ID NO: 21 - RNA, 22 nt, synthetic construct
  source 1..22: mol_type = other RNA
  modified_base 1: mod_base = OTHER; note = 2'-a GalNAc clicked cytidine
  modified_base 2: mod_base = OTHER; note = Residue has a 2'-deoxy-2'-fluoro (2'-F) ribosugar modification
  modified_base 3: mod_base = gm
  modified_base 4: mod_base = OTHER; note = Residue has a 2'-deoxy-2'-fluoro (2'-F) ribosugar modification
  modified_base 5: mod_base = gm
  modified_base 6: mod_base = OTHER; note = Residue has a 2'-deoxy-2'-fluoro (2'-F) ribosugar modification
  modified_base 7: mod_base = gm
  modified_base 8: mod_base = OTHER; note = Residue has a 2'-deoxy-2'-fluoro (2'-F) ribosugar modification
  modified_base 9: mod_base = gm
  modified_base 10: mod_base = OTHER; note = Residue has a 2'-deoxy-2'-fluoro (2'-F) ribosugar modification
  modified_base 11: mod_base = gm
  modified_base 12: mod_base = OTHER; note = Residue has a 2'-deoxy-2'-fluoro (2'-F) ribosugar modification
  modified_base 13: mod_base = um
  modified_base 14: mod_base = OTHER; note = Residue has a 2'-deoxy-2'-fluoro (2'-F) ribosugar modification
  modified_base 15: mod_base = um
  modified_base 16: mod_base = OTHER; note = Residue has a 2'-deoxy-2'-fluoro (2'-F) ribosugar modification
  modified_base 17: mod_base = um
  modified_base 18: mod_base = OTHER; note = Residue has a 2'-deoxy-2'-fluoro (2'-F) ribosugar modification
  modified_base 19: mod_base = cm
  modified_base 20: mod_base = OTHER; note = Residue has a 2'-deoxy-2'-fluoro (2'-F) ribosugar modification
  modified_base 21: mod_base = cm
  modified_base 22: mod_base = OTHER; note = Residue has a 2'-deoxy-2'-fluoro (2'-F) ribosugar modification
ctgggagagggttgtttactcc

SEQ ID NO: 22 - RNA, 23 nt, synthetic construct
  source 1..23: mol_type = other RNA
  modified_base 1: mod_base = OTHER; note = Residue has a 2'-deoxy-2'-fluoro (2'-F) ribosugar modification
  modified_base 2: mod_base = um
  modified_base 3: mod_base = OTHER; note = Residue has a 2'-deoxy-2'-fluoro (2'-F) ribosugar modification
  modified_base 4: mod_base = gm
  modified_base 5: mod_base = OTHER; note = Residue has a 2'-deoxy-2'-fluoro (2'-F) ribosugar modification
  modified_base 6: mod_base = OTHER; note = Residue is 2'-O-methylated
  modified_base 7: mod_base = OTHER; note = Residue has a 2'-deoxy-2'-fluoro (2'-F) ribosugar modification
  modified_base 8: mod_base = OTHER; note = Residue is 2'-O-methylated
  modified_base 9: mod_base = OTHER; note = Residue has a 2'-deoxy-2'-fluoro (2'-F) ribosugar modification
  modified_base 10: mod_base = gm
  modified_base 11: mod_base = OTHER; note = Residue has a 2'-deoxy-2'-fluoro (2'-F) ribosugar modification
  modified_base 12: mod_base = um
  modified_base 13: mod_base = OTHER; note = Residue has a 2'-deoxy-2'-fluoro (2'-F) ribosugar modification
  modified_base 14: mod_base = gm
  modified_base 15: mod_base = OTHER; note = Residue has a 2'-deoxy-2'-fluoro (2'-F) ribosugar modification
  modified_base 16: mod_base = um
  modified_base 17: mod_base = OTHER; note = Residue has a 2'-deoxy-2'-fluoro (2'-F) ribosugar modification
  modified_base 18: mod_base = OTHER; note = Residue is 2'-O-methylated
  modified_base 19: mod_base = OTHER; note = Residue has a 2'-deoxy-2'-fluoro (2'-F) ribosugar modification
  modified_base 20: mod_base = um
  modified_base 21: mod_base = OTHER; note = Residue has a 2'-deoxy-2'-fluoro (2'-F) ribosugar modification
  modified_base 22: mod_base = OTHER; note = 2'-a GalNAc clicked cytidine
  modified_base 23: mod_base = um
ctgggagagggttgtttactcct

SEQ ID NO: 23 - RNA, 23 nt, synthetic construct
  source 1..23: mol_type = other RNA
  modified_base 1: mod_base = OTHER; note = 2'-a GalNAc clicked cytidine
  modified_base 2: mod_base = um
  modified_base 3: mod_base = OTHER; note = Residue has a 2'-deoxy-2'-fluoro (2'-F) ribosugar modification
  modified_base 4: mod_base = gm
  modified_base 5: mod_base = OTHER; note = Residue has a 2'-deoxy-2'-fluoro (2'-F) ribosugar modification
  modified_base 6: mod_base = OTHER; note = Residue is 2'-O-methylated
  modified_base 7: mod_base = OTHER; note = Residue has a 2'-deoxy-2'-fluoro (2'-F) ribosugar modification
  modified_base 8: mod_base = OTHER; note = Residue is 2'-O-methylated
  modified_base 9: mod_base = OTHER; note = Residue has a 2'-deoxy-2'-fluoro (2'-F) ribosugar modification
  modified_base 10: mod_base = gm
  modified_base 11: mod_base = OTHER; note = Residue has a 2'-deoxy-2'-fluoro (2'-F) ribosugar modification
  modified_base 12: mod_base = um
  modified_base 13: mod_base = OTHER; note = Residue has a 2'-deoxy-2'-fluoro (2'-F) ribosugar modification
  modified_base 14: mod_base = gm
  modified_base 15: mod_base = OTHER; note = Residue has a 2'-deoxy-2'-fluoro (2'-F) ribosugar modification
  modified_base 16: mod_base = um
  modified_base 17: mod_base = OTHER; note = Residue has a 2'-deoxy-2'-fluoro (2'-F) ribosugar modification
  modified_base 18: mod_base = OTHER; note = Residue is 2'-O-methylated
  modified_base 19: mod_base = OTHER; note = Residue has a 2'-deoxy-2'-fluoro (2'-F) ribosugar modification
  modified_base 20: mod_base = um
  modified_base 21: mod_base = OTHER; note = Residue has a 2'-deoxy-2'-fluoro (2'-F) ribosugar modification
  modified_base 22: mod_base = OTHER; note = 2'-a GalNAc clicked cytidine
  modified_base 23: mod_base = um
ctgggagagggttgtttactcct

SEQ ID NO: 24 - RNA, 22 nt, synthetic construct
  source 1..22: mol_type = other RNA
  modified_base 1: mod_base = OTHER; note = 2'-a GalNAc clicked cytidine
  modified_base 2: mod_base = OTHER; note = Residue has a 2'-deoxy-2'-fluoro (2'-F) ribosugar modification
  modified_base 3: mod_base = gm
  modified_base 4: mod_base = OTHER; note = Residue has a 2'-deoxy-2'-fluoro (2'-F) ribosugar modification
  modified_base 5: mod_base = gm
  modified_base 6: mod_base = OTHER; note = Residue has a 2'-deoxy-2'-fluoro (2'-F) ribosugar modification
  modified_base 7: mod_base = gm
  modified_base 8: mod_base = OTHER; note = Residue has a 2'-deoxy-2'-fluoro (2'-F) ribosugar modification
  modified_base 9: mod_base = OTHER; note = Residue is 2'-O-methylated
  modified_base 10: mod_base = OTHER; note = Residue has a 2'-deoxy-2'-fluoro (2'-F) ribosugar modification
  modified_base 11: mod_base = gm
  modified_base 12: mod_base = OTHER; note = Residue has a 2'-deoxy-2'-fluoro (2'-F) ribosugar modification
  modified_base 13: mod_base = um
  modified_base 14: mod_base = OTHER; note = Residue has a 2'-deoxy-2'-fluoro (2'-F) ribosugar modification
  modified_base 15: mod_base = um
  modified_base 16: mod_base = OTHER; note = Residue has a 2'-deoxy-2'-fluoro (2'-F) ribosugar modification
  modified_base 17: mod_base = um
  modified_base 18: mod_base = OTHER; note = Residue has a 2'-deoxy-2'-fluoro (2'-F) ribosugar modification
  modified_base 19: mod_base = cm
  modified_base 20: mod_base = OTHER; note = Residue has a 2'-deoxy-2'-fluoro (2'-F) ribosugar modification
  modified_base 21: mod_base = cm
  modified_base 22: mod_base = OTHER; note = Residue has a 2'-deoxy-2'-fluoro (2'-F) ribosugar modification
ctgggagaaggctgtttactct

SEQ ID NO: 25 - RNA, 23 nt, synthetic construct
  source 1..23: mol_type = other RNA
  modified_base 1: mod_base = OTHER; note = Residue has a 2'-deoxy-2'-fluoro (2'-F) ribosugar modification
  modified_base 2: mod_base = um
  modified_base 3: mod_base = OTHER; note = Residue has a 2'-deoxy-2'-fluoro (2'-F) ribosugar modification
  modified_base 4: mod_base = gm
  modified_base 5: mod_base = OTHER; note = Residue has a 2'-deoxy-2'-fluoro (2'-F) ribosugar modification
  modified_base 6: mod_base = OTHER; note = Residues is 2'-O-methylated
  modified_base 7: mod_base = OTHER; note = Residue has a 2'-deoxy-2'-fluoro (2'-F) ribosugar modification
  modified_base 8: mod_base = OTHER; note = Residue is 2'-O-methylated
  modified_base 9: mod_base = OTHER; note = Residue has a 2'-deoxy-2'-fluoro (2'-F) ribosugar modification
  modified_base 10: mod_base = gm
  modified_base 11: mod_base = OTHER; note = Residue has a 2'-deoxy-2'-fluoro (2'-F) ribosugar modification
  modified_base 12: mod_base = um
  modified_base 13: mod_base = OTHER; note = Residue has a 2'-deoxy-2'-fluoro (2'-F) ribosugar modification
  modified_base 14: mod_base = gm
  modified_base 15: mod_base = OTHER; note = Residue has a 2'-deoxy-2'-fluoro (2'-F) ribosugar modification
  modified_base 16: mod_base = um
  modified_base 17: mod_base = OTHER; note = Residue has a 2'-deoxy-2'-fluoro (2'-F) ribosugar modification
  modified_base 18: mod_base = OTHER; note = Residue is 2'-O-methylated
  modified_base 19: mod_base = OTHER; note = 2'-a GalNAc clicked cytidine
  modified_base 20: mod_base = OTHER; note = Residue has a 2'-deoxy-2'-fluoro (2'-F) ribosugar modification
  modified_base 21: mod_base = OTHER; note = 2'-a GalNAc clicked cytidine
  modified_base 22: mod_base = OTHER; note = 2'-a GalNAc clicked cytidine
  modified_base 23: mod_base = um
ctgggagagggttgtttactcct

SEQ ID NO: 26 - RNA, 23 nt, synthetic construct
  source 1..23: mol_type = other RNA
  modified_base 1: mod_base = OTHER; note = 2'-a GalNAc clicked cytidine
  modified_base 2: mod_base = OTHER; note = Residue has a 2'-deoxy-2'-fluoro (2'-F) ribosugar modification
  modified_base 3: mod_base = gm
  modified_base 4: mod_base = OTHER; note = Residue has a 2'-deoxy-2'-fluoro (2'-F) ribosugar modification
  modified_base 5: mod_base = gm
  modified_base 6: mod_base = OTHER; note = Residue has a 2'-deoxy-2'-fluoro (2'-F) ribosugar modification
  modified_base 7: mod_base = gm
  modified_base 8: mod_base = OTHER; note = Residue has a 2'-deoxy-2'-fluoro (2'-F) ribosugar modification
  modified_base 9: mod_base = gm
  modified_base 10: mod_base = OTHER; note = Residue has a 2'-deoxy-2'-fluoro (2'-F) ribosugar modification
  modified_base 11: mod_base = gm
  modified_base 12: mod_base = OTHER; note = Residue has a 2'-deoxy-2'-fluoro (2'-F) ribosugar modification
  modified_base 13: mod_base = um
  modified_base 14: mod_base = OTHER; note = Residue has a 2'-deoxy-2'-fluoro (2'-F) ribosugar modification
  modified_base 15: mod_base = um
  modified_base 16: mod_base = OTHER; note = Residue has a 2'-deoxy-2'-fluoro (2'-F) ribosugar modification
  modified_base 17: mod_base = um
  modified_base 18: mod_base = OTHER; note = Residue has a 2'-deoxy-2'-fluoro (2'-F) ribosugar modification
  modified_base 19: mod_base = OTHER; note = 2'-a GalNAc clicked cytidine
  modified_base 20: mod_base = OTHER; note = Residue has a 2'-deoxy-2'-fluoro (2'-F) ribosugar modification
  modified_base 21: mod_base = OTHER; note = 2'-a GalNAc clicked cytidine
  modified_base 22: mod_base = OTHER; note = 2'-a GalNAc clicked cytidine
  modified_base 23: mod_base = um
ctgggagagggttgtttactcct

SEQ ID NO: 27 - RNA, 22 nt, synthetic construct
  source 1..22: mol_type = other RNA
  modified_base 1: mod_base = OTHER; note = Residue has a 2'-deoxy-2'-fluoro (2'-F) ribosugar modification
  modified_base 2: mod_base = um
  modified_base 3: mod_base = OTHER; note = Residue has a 2'-deoxy-2'-fluoro (2'-F) ribosugar modification
  modified_base 4: mod_base = gm
  modified_base 5: mod_base = OTHER; note = Residue has a 2'-deoxy-2'-fluoro (2'-F) ribosugar modification
  modified_base 6: mod_base = OTHER; note = Residue is 2'-O-methylated
  modified_base 7: mod_base = OTHER; note = Residue has a 2'-deoxy-2'-fluoro (2'-F) ribosugar modification
  modified_base 8: mod_base = OTHER; note = Residue is 2'-O-methylated
  modified_base 9: mod_base = OTHER; note = Residue has a 2'-deoxy-2'-fluoro (2'-F) ribosugar modification
  modified_base 10: mod_base = gm
  modified_base 11: mod_base = OTHER; note = Residue has a 2'-deoxy-2'-fluoro (2'-F) ribosugar modification
  modified_base 12: mod_base = OTHER; note = 2'-a GalNAc clicked cytidine
  modified_base 13: mod_base = um
  modified_base 14: mod_base = OTHER; note = Residue has a 2'-deoxy-2'-fluoro (2'-F) ribosugar modification
  modified_base 15: mod_base = um
  modified_base 16: mod_base = OTHER; note = Residue has a 2'-deoxy-2'-fluoro (2'-F) ribosugar modification
  modified_base 17: mod_base = um
  modified_base 18: mod_base = OTHER; note = Residue has a 2'-deoxy-2'-fluoro (2'-F) ribosugar modification
  modified_base 19: mod_base = OTHER; note = 2'-a GalNAc clicked cytidine
  modified_base 20: mod_base = um
  modified_base 21: mod_base = OTHER; note = 2'-a GalNAc clicked cytidine
  modified_base 22: mod_base = OTHER; note = Residue has a 2'-deoxy-2'-fluoro (2'-F) ribosugar modification
ctgggagaaggctgtttactct

SEQ ID NO: 28 - RNA, 22 nt, synthetic construct
  source 1..22: mol_type = other RNA
  modified_base 1: mod_base = OTHER; note = 2'-a GalNAc clicked cytidine
  modified_base 2: mod_base = OTHER; note = Residue has a 2'-deoxy-2'-fluoro (2'-F) ribosugar modification
  modified_base 3: mod_base = gm
  modified_base 4: mod_base = OTHER; note = Residue has a 2'-deoxy-2'-fluoro (2'-F) ribosugar modification
  modified_base 5: mod_base = gm
  modified_base 6: mod_base = OTHER; note = Residue has a 2'-deoxy-2'-fluoro (2'-F) ribosugar modification
  modified_base 7: mod_base = gm
  modified_base 8: mod_base = OTHER; note = Residue has a 2'-deoxy-2'-fluoro (2'-F) ribosugar modification
  modified_base 9: mod_base = OTHER; note = Residue is 2'-O-methylated
  modified_base 10: mod_base = OTHER; note = Residue has a 2'-deoxy-2'-fluoro (2'-F) ribosugar modification
  modified_base 11: mod_base = gm
  modified_base 12: mod_base = OTHER; note = 2'-a GalNAc clicked cytidine
  modified_base 13: mod_base = OTHER; note = Residue has a 2'-deoxy-2'-fluoro (2'-F) ribosugar modification
  modified_base 14: mod_base = gm
  modified_base 15: mod_base = OTHER; note = Residue has a 2'-deoxy-2'-fluoro (2'-F) ribosugar modification
  modified_base 16: mod_base = um
  modified_base 17: mod_base = OTHER; note = Residue has a 2'-deoxy-2'-fluoro (2'-F) ribosugar modification
  modified_base 18: mod_base = OTHER; note = Residue is 2'-O-methylated
  modified_base 19: mod_base = OTHER; note = 2'-a GalNAc clicked cytidine
  modified_base 20: mod_base = OTHER; note = Residue has a 2'-deoxy-2'-fluoro (2'-F) ribosugar modification
  modified_base 21: mod_base = OTHER; note = 2'-a GalNAc clicked cytidine
  modified_base 22: mod_base = um
ctgggagaaggctgtttactct

SEQ ID NO: 29 - RNA, 24 nt, synthetic construct
  source 1..24: mol_type = other RNA
  modified_base 1: mod_base = OTHER; note = 2'-a GalNAc clicked cytidine
  modified_base 2: mod_base = OTHER; note = 2'-a GalNAc clicked cytidine
  modified_base 3: mod_base = OTHER; note = 2'-a GalNAc clicked cytidine
  modified_base 4: mod_base = OTHER; note = Residue has a 2'-deoxy-2'-fluoro (2'-F) ribosugar modification
  modified_base 5: mod_base = gm
  modified_base 6: mod_base = OTHER; note = Residue has a 2'-deoxy-2'-fluoro (2'-F) ribosugar modification
  modified_base 7: mod_base = gm
  modified_base 8: mod_base = OTHER; note = Residue has a 2'-deoxy-2'-fluoro (2'-F) ribosugar modification
  modified_base 9: mod_base = gm
  modified_base 10: mod_base = OTHER; note = Residue has a 2'-deoxy-2'-fluoro (2'-F) ribosugar modification
  modified_base 11: mod_base = gm
  modified_base 12: mod_base = OTHER; note = Residue has a 2'-deoxy-2'-fluoro (2'-F) ribosugar modification
  modified_base 13: mod_base = gm
  modified_base 14: mod_base = OTHER; note = Residue has a 2'-deoxy-2'-fluoro (2'-F) ribosugar modification
  modified_base 15: mod_base = um
  modified_base 16: mod_base = OTHER; note = Residue has a 2'-deoxy-2'-fluoro (2'-F) ribosugar modification
  modified_base 17: mod_base = um
  modified_base 18: mod_base = OTHER; note = Residue has a 2'-deoxy-2'-fluoro (2'-F) ribosugar modification
  modified_base 19: mod_base = um
  modified_base 20: mod_base = OTHER; note = Residue has a 2'-deoxy-2'-fluoro (2'-F) ribosugar modification
  modified_base 21: mod_base = cm
  modified_base 22: mod_base = OTHER; note = Residue has a 2'-deoxy-2'-fluoro (2'-F) ribosugar modification
  modified_base 23: mod_base = cm
  modified_base 24: mod_base = OTHER; note = Residue has a 2'-deoxy-2'-fluoro (2'-F) ribosugar modification
ccctgggagagggttgtttactcc

SEQ ID NO: 30 - RNA, 24 nt, synthetic construct
  source 1..24: mol_type = other RNA
  modified_base 1: mod_base = OTHER; note = 2'-a GalNAc clicked cytidine
  modified_base 2: mod_base = OTHER; note = 2'-a GalNAc clicked cytidine
  modified_base 3: mod_base = OTHER; note = 2'-a GalNAc clicked cytidine
  modified_base 4: mod_base = OTHER; note = Residue has a 2'-deoxy-2'-fluoro (2'-F) ribosugar modification
  modified_base 5: mod_base = gm
  modified_base 6: mod_base = OTHER; note = Residue has a 2'-deoxy-2'-fluoro (2'-F) ribosugar modification
  modified_base 7: mod_base = gm
  modified_base 8: mod_base = OTHER; note = Residue has a 2'-deoxy-2'-fluoro (2'-F) ribosugar modification
  modified_base 9: mod_base = gm
  modified_base 10: mod_base = OTHER; note = Residue has a 2'-deoxy-2'-fluoro (2'-F) ribosugar modification
  modified_base 11: mod_base = OTHER; note = Residue is 2'-O-methylated
  modified_base 12: mod_base = OTHER; note = Residue has a 2'-deoxy-2'-fluoro (2'-F) ribosugar modification
  modified_base 13: mod_base = gm
  modified_base 14: mod_base = OTHER; note = Residue has a 2'-deoxy-2'-fluoro (2'-F) ribosugar modification
  modified_base 15: mod_base = um
  modified_base 16: mod_base = OTHER; note = Residue has a 2'-deoxy-2'-fluoro (2'-F) ribosugar modification
  modified_base 17: mod_base = um
  modified_base 18: mod_base = OTHER; note = Residue has a 2'-deoxy-2'-fluoro (2'-F) ribosugar modification
  modified_base 19: mod_base = um
  modified_base 20: mod_base = OTHER; note = Residue has a 2'-deoxy-2'-fluoro (2'-F) ribosugar modification
  modified_base 21: mod_base = cm
  modified_base 22: mod_base = OTHER; note = Residue has a 2'-deoxy-2'-fluoro (2'-F) ribosugar modification
  modified_base 23: mod_base = cm
  modified_base 24: mod_base = OTHER; note = Residue has a 2'-deoxy-2'-fluoro (2'-F) ribosugar modification
ccctgggagaaggctgtttactct

SEQ ID NO: 31 - RNA, 24 nt, synthetic construct
  source 1..24: mol_type = other RNA
  modified_base 1: mod_base = OTHER; note = 2'-a GalNAc clicked cytidine
  modified_base 2: mod_base = OTHER; note = 2'-a GalNAc clicked cytidine
  modified_base 3: mod_base = OTHER; note = 2'-a GalNAc clicked cytidine
  modified_base 4: mod_base = um
  modified_base 5: mod_base = gm
  modified_base 6: mod_base = gm
  modified_base 7: mod_base = gm
  modified_base 8: mod_base = OTHER; note = Residue is 2'-O-methylated
  modified_base 9: mod_base = gm
  modified_base 10: mod_base = OTHER; note = Residue is 2'-O-methylated
  modified_base 11: mod_base = OTHER; note = Residue is 2'-O-methylated
  modified_base 12: mod_base = gm
  modified_base 13: mod_base = gm
  modified_base 14: mod_base = cm
  modified_base 15: mod_base = um
  modified_base 16: mod_base = gm
  modified_base 17: mod_base = um
  modified_base 18: mod_base = um
  modified_base 19: mod_base = um
  modified_base 20: mod_base = OTHER; note = Residue is 2'-O-methylated
  modified_base 21: mod_base = cm
  modified_base 22: mod_base = um
  modified_base 23: mod_base = cm
  modified_base 24: mod_base = um
ccctgggagaaggctgtttactct

SEQ ID NO: 32 - DNA, 21 bp, synthetic construct
  source 1..21: mol_type = other DNA
tgtggccttactatggaggaa

(19) United States
(12) Patent Application Publication
(10) Pub. No.: US 2023/0407302 A1
(43) Pub. Date: Dec. 21, 2023

(54) NOVEL EFFICACIOUS MICRORNA-30C ANALOGS REDUCE APOLIPOPROTEIN B SECRETION IN HUMAN LIVER CELLS

(71) Applicants: New York University, New York, NY (US); The Research Foundation for the State University of New York, Albany, NY (US); The United States Government as represented by the Department of Veterans Affairs, Washington, DC (US)

(72) Inventors: Mahmood Hussain, Woodbury, NY (US); Jia Sheng, Schenectady, NY (US); Pradeep Kumar Yadav, Varanasi (IN); Phensinee Haruehanroengra, Billerica, MA (US)

(21) Appl. No.: 18/164,699

(22) Filed: Feb. 6, 2023

Related U.S. Application Data
(60) Provisional application No. 63/306,726, filed on Feb. 4, 2022.

(51) Int. Cl. C12N 15/113

(52) U.S. Cl. CPC C12N 15/113; C12N 2310/141; C12N 2310/321; C12N 2310/351; C12N 2310/315

(57) ABSTRACT

Provided are potent miR-30c analogs that can be delivered to hepatoma cells without the aid of viral vectors and lipid emulsions. The miR-30c analogs contain an unmodified active sense strand and a modified passenger strand to enhance the stability and uptake of miR-30c by hepatoma cells. The miR-30c analogs are for use in treatment of hyperlipidemias and cardiovascular diseases.

### CROSS REFERENCE TO RELATED APPLICATION

This application claims priority to U.S. provisional application No. 63/306,726, filed Feb. 4, 2022, the entire disclosure of which is incorporated herein by reference.

### STATEMENT REGARDING FEDERALLY SPONSORED RESEARCH

This invention was made with government support under grant numbers DK121490, HL137202, and HD094778, awarded by the National Institutes of Health, grant number BX004113 awarded by the United States Department of Veterans Affairs, and grant numbers CHE1845486 and MCB1715234 awarded by the National Science Foundation. The government has certain rights in the invention.

### SEQUENCE LISTING

The instant application contains a Sequence Listing which has been submitted in .xml format and is hereby incorporated by reference in its entirety. Said .xml file is named “058636_00575_ST26_2.xml”, was created on Apr. 18, 2023, and is 267,332 bytes in size.

### RELATED INFORMATION

Atherosclerosis, hardening of the arteries after lipid deposition, is the leading cause of morbidity and mortality in the US and worldwide. High plasma cholesterol levels are a major risk factor for atherosclerosis. Cholesterol in the circulation is carried primarily by apolipoprotein B (apoB) containing lipoproteins. Remarkable advances have been made in lowering plasma cholesterol and reducing death by 30%-40% through treatments with statins and proprotein convertase subtilis/kexin type 9 inhibitors (1-4). Despite the availability of these drugs, an unmet need for new lipid lowering therapies remains, because some patients do not achieve the desirable cholesterol lowering with statins (5); a substantial proportion of patients experience unmanageable adverse effects (6,7); and statins and PCSK9 antibodies are not useful in treating patients with homozygous familial hypercholesterolemia and low density lipoprotein receptor null mutations (8,9). Therefore, a need exists to identify safer methods of lowering plasma lipids that can be used independently of or in combination with statins and other available drugs.

Statins, inhibitors of hydroxyl-methyl-glutaryl-coenzyme A reductase, lower plasma lipids by increasing the hepatic expression of low-density lipoprotein receptors and decreasing cholesterol synthesis. A complementary approach involves inhibiting the assembly and secretion of lipoproteins to limit their entry into the circulation. Lipoprotein assembly requires two proteins: the structural protein apoB and the chaperone microsomal triglyceride transfer protein (MTP). MTP physically interacts with and transfers lipids in the endoplasmic reticulum to nascent apoB, and assists in the formation and maturation of lipoprotein particles for secretion (10,11). MTP has long been a drug target for lowering plasma lipids, as its biochemical activity of transferring lipids can be easily measured in laboratory settings. Several pharmaceutical companies have developed drugs that potently inhibit MTP activity and lower plasma lipids (12,13). However, these drugs increase hepatic lipids and plasma transaminases (14-16). One MTP inhibitor, lomitapide, has been approved for the treatment of homozygous familial hypercholesterolemia on a restricted protocol and carries label warnings for hepatic steatosis (17,18). Hence, a need remains for agents that can reduce levels of MTP and plasma lipids without causing steatosis.

MicroRNAs (miRs) are endogenous gene products ˜22 nucleotides in length that regulate gene expression at the post-transcriptional level. They interact with the 3′-untranslated regions of target mRNAs and decrease protein synthesis by enhancing mRNA degradation and/or interfering with translation (19,20). Currently, several miR-based drugs are in clinical trials for the treatment of atherosclerosis, heart failure, diabetes, and hepatitis C viral infection, and are expected to be possible treatments in the future (21,22). A major hurdle in the development of miR therapeutics involving chemical modifications is the loss of mRNA silencing. Consequently, several methods of delivery have been devised, including viral vectors and neutral lipid emulsions (21).

MiR-30c is a small (23 nucleotide), double stranded, non-coding RNA. The 5′-physiologically active sense strand interacts with different mRNAs and subsequently modulates the synthesis of various proteins (23,24). MiR-30c is derived from the products of two genes (MIR30C1 and MIR30C2) in humans and mice (23). The primary transcripts of these genes, pri-miR-30c-1 and pri-miR-30c-2, show 56.7% and 58.5% similarity, respectively, between humans and mice. These transcripts are processed in the nucleus and exported to the cytoplasm as pre-miR-30c-1 and pre-miR-30c-2. Pre-miR-30c-1 is highly similar between humans and mice (98.87% similarity); in contrast, pre-miR-30c-2 is 82.1% similar between both species. These pre-miRs are further processed in the cytoplasm, thus resulting in the production of mature miR-30c with identical 5′-strands (miR-30c-5p) that are conserved in humans and mice. The 3′-strands (miR-30c-3p) derived from the two genes are slightly different, but are conserved in humans and mice.

It has been reported that overexpression of miR-30c significantly reduces MTP activity, whereas overexpression of its corresponding anti-miR elevates MTP activity in Huh-7 human hepatoma cells and human primary hepatocytes (25-27). Furthermore, miR-30c significantly reduces apoB secretion, whereas anti-miR-30c increases apoB secretion without affecting apolipoprotein A1 (apoA1) secretion in these cells. Mechanistic studies revealed that miR-30c decreases MTP activity by interacting with and degrading RNA at the post-transcriptional level (25,28,29). MiR-interacts with MTP mRNA involving both the seed and supplementary sites (26).

To investigate whether miR-30c regulates MTP activity and plasma lipids in vivo, we intravenously transduced male C57BL/6 mice with lentiviruses for the expression of control, miR-30c, or anti-miR-30c and then fed them a Western diet (25). MiR-30c decreased hepatic MTP expression, plasma cholesterol, and hepatic lipoprotein production. Despite reductions in plasma cholesterol, increases in hepatic lipids and plasma transaminases in miR-30c expressing mice were not detected. Because viral therapy is formidable, we intravenously injected miR-30c analogs complexed with lipid emulsions (28). These emulsions enabled delivery of miR-30c to the liver and diminished diet-induced hypercholesterolemia in the mice. Furthermore, we found that miR-30c mimic significantly reduces hypercholesterolemia and atherosclerosis in Apoe^{-/-} mice (28). Subsequent studies demonstrated that miR-30c also decreases plasma cholesterol in diabetic ob/ob and db/db mice and in Western-diet fed LdIr^{-/-} mice, but has no effect on plasma triglycerides, glucose, and transaminases (29). These studies have indicated that the hepatic expression of miR-30c decreases plasma cholesterol, hepatic lipid synthesis, and atherosclerosis without causing steatosis seen with MTP inhibitors. In the studies summarized above (25,28,29), miR-30c was injected intravenously as lentiviruses or complexed with lipid emulsions. Because these approaches can be expensive and difficult for therapeutic interventions, there remains a need for improved compositions for use in to reducing apoB secretion without affecting apoA1 secretion and hepatic lipid accumulation in a viral vector and liposome free manner. The present disclosure is pertinent to this need.

### BRIEF SUMMARY

The present disclosure provides, among other aspects, viral vector and liposome free compositions comprising new and potent miR-30c analogs, and methods of using the potent miR-30c analogs and compositions. The miR-30c analogs are in some embodiments provided as a double stranded polynucleotide complex. The double stranded polynucleotide complex comprises a first strand which optionally comprises no nucleotide modifications and a second strand comprising nucleotide modifications that forms the double stranded polynucleotide complex with the first strand. The second modified second strands include the following:

| | | | | SEQ ID |
|---|---|---|---|---|
| | | | | NO: |
| miR30c-C1 | C1 | miR-30c-1-3p | (pC)•U•gGgAgAgGgUuGuUuAcUc•C | 1 |
| miR30c-C2 | C2 | miR-30c-1-3p | (pC)•UgGgAgAgGgUuGuUuAc•U•c•C | 2 |
| miR30c-C3 | C3 | miR-30c-1-3p | (pc)•U•gGgAgAgGgUuGuUuAc•U•c•C | 3 |
| miR30c-C4 | C4 | miR-30c-1-3p | GgGaGaGgGuUgUuUaCuC(pC)•u | 4 |
| miR30c-C5 | C5 | miR-30c-1-3p | GgGaGaGgGuUgUuUaCu•C•(pC)•u | 5 |
| miR30c-C6 | C6 | miR-30c-1-3p | C•u•GgGaGaGgGuUgUuUaCuC(pC)u | 6 |

wherein upper case letters signify a 2′-deoxy-2′-fluoro (2′-F) ribosugar modification; lower case letters signify a 2′-O-methyl (2′-OMe) ribosugar modification; pC signifies 2′-a GalNAc clicked cytidine, and the • symbol signifies a phosphorothioate linkage. In one example the second strand comprises the C2 strand. In embodiments, the described complex that comprises the C2 strand exhibits one or more improved properties relative to a complex that comprises the C1, C3, C4, C5, or C6 strand.

The disclosure also provides a method for inhibiting apoB secretion from cells by introducing into the cells a described double stranded polynucleotide complex in a composition that does not include any viral expression vector or liposomal components. In embodiments the described method does not reduce apoA1 secretion, or reduces apoA1 secretion less than a control value obtained from introducing into the cells a double stranded polynucleotide complex that does comprise the C2 strand.

### BRIEF DESCRIPTION OF THE FIGURES

FIG. 1. MiR-30c sequences and representative RNA modifications. (A and B) The two double stranded mature miR-30c sequences derived from two independent genes are shown. The two passenger strands, miR-30c-1 and miR-30c-2, are in red. The common miR-30c-5p strand is in black. (C) Different chemically modified nucleotides used in the synthesis of modified miR-30c passenger strands. (D) Synthesis of GalNAc cytidine.

FIG. 2. Thermal stability and circular dichroism spectra of modified miR-30c duplexes. (A) Melting temperature and (B) CD spectra of native miR-30c, and 2′-OMe modified miR-30c-1-3p and miR-30c-2-3p duplexed with native miR-30c-strand.

FIG. 3. Activity of 2′-OMe modified miR-30c-1 and miR-30c-2 in Huh-7 cells. Human hepatoma cells (Huh-7) were reverse transfected with 100 nM of 2″-OMe modified miR-30c analogs complexed with Lipofectamine RNAiMax transfection at a ratio of 3:1. For the control, cells were treated only with Lipofectamine RNAiMax (no mimics). Forty-eight hours after transfection with 2′-OMe-30c-1 and 2′-OMe-30c-2 analogs, media were collected and used to measure apoB (A and B) and apoA1 (C and D) concentrations. Data are representative of three independent experiments. Significance was determined at p<0.05 (*) with Student's t test.

FIG. 4. Effects of different miR-30c series A analogs on apoB secretion in Huh-7 cells. (A) ApoB secretion in Huh-7 cells transfected with control (Ctrl) and different miR-30c analogs (100 nM), as described in Table 1, with Lipofectamine RNAiMax. MiR-30c (30c) was used a positive control. (B) ApoA1 secretion in Huh-7 cells transfected with control (Ctrl) and different miR-30c analogs (100 nM) with Lipofectamine RNAiMax. (C) ApoB secretion in Huh-7 cells transfected with Ctrl and different miR-30c analogs (1000 nM) without Lipofectamine RNAiMax. (D) ApoA1 secretion in Huh-7 cells transfected with Ctrl and different miR-30c analogs without Lipofectamine RNAiMax. The apoB and apoA1 concentrations were measured by ELISA in the collected media. Data are representative of four independent experiments. Significance was determined at p<0.0001 (****) and p<0.001 (***) with ANOVA.

FIG. 5. Synthesis of 3-chloropropyl GalNAc 2 and GalNAcαProN3 3. (A) 3-Chloropropyl GalNAc 2 and GalNAcαProN3 3 were synthesized as described in the methods. (B and C) ¹H NMR spectra of 3-chloropropyl GalNAc 2 and GalNAcαProN3 3 are shown. (D) Analytical 15% polyacrylamide 8 M urea gel electrophoresis of antisense-oligonucleotide (ASO) in lane 1, post-clicked GalNAc-ASO in lane 2, and reference GalNAc-ASO in lane 3.

FIG. 6. Effects of various GalNAc-modified miR-30c analogs on apoB secretion in Huh-7 human hepatoma cells. (A) ApoB secretion and (B) apoA1 secretion in Huh-7 cells transfected with control (Ctrl) and different miR-30c series B analogs (Table 2) with Lipofectamine RNAiMax. MiR-30c (30c) was used as a positive control. (C) ApoB and (D) apoA1 secretion in Huh-7 cells transfected with Ctrl and different miR-30c analogs without Lipofectamine RNAiMax. The apoB and apoA1 concentrations in the media were measured with ELISA. Data are representative of four independent experiments. Significance was determined at p<0.0001 (****) and p<0.001 (***) with ANOVA.

FIG. 7. Effects of GalNAc-modified B1 and B2 analogs on apoB secretion and MTP activity in Huh-7 cells. (A) ApoB and (B) apoA1 secretion (%) in the culture medium of Huh-7 cells transfected with increasing amounts of miR-30c, or B1 or B2 analog without Lipofectamine RNAiMax. Non-transfected cells were used as controls. Data are representative of two independent experiments. (C-F) cells were exposed to 250 nM of miR-30c or B1 without transfection reagent. After 48 h, the cells were used to measure (C) miR-30c levels, (D) MTP mRNA, (E) MTP activity, and (F) MTP protein. Data are representative of three independent experiments. Western blot analysis is from one representative experiment. After transfer, the blot was cut into two parts; the top was used to detect MTP, and the bottom was used to detect β-actin as a loading control. Significance was determined at p<0.05 (*) and p<0.0001 (****) with Student's t test and ANOVA.

FIG. 8. Effects of various phosphorothioate-linked miR-30c analogs on apoB secretion in Huh-7 human hepatoma cells. (A) ApoB and (B) apoA1 secretion in Huh-7 cells transfected with Ctrl and different synthetic miR-30c series C analogs (Table 3) with Lipofectamine RNAiMax. MiR-30c (30c) was used as a positive control. (C) ApoB and (D) apoA1 secretion in Huh-7 cells transfected with Ctrl and different miR-30c analogs without Lipofectamine RNAiMax. Data are representative of three independent experiments. Relative (E) apoB and (F) apoA1 secretion (%) in culture medium of Huh-7 cells transfected with increasing amounts of miR-30c or analog without Lipofectamine/RNAiMax. Non-transfected cells were used as a control. Data are representative of two independent experiments. Significance was determined at p<0.0001 (****), p<0.001 (***), and p<0.01 (**) with ANOVA.

### DETAILED DESCRIPTION

Unless defined otherwise herein, all technical and scientific terms used herein have the same meaning as commonly understood by one of ordinary skill in the art to which this disclosure pertains. The disclosure includes the following abbreviations: ApoA1, apolipoprotein A1; ApoB, apolipoprotein B; CD, circular dichroism; Ctrl, control; DMEM, Dulbecco's modified Eagle's medium; FBS, fetal bovine serum; GalNac, N-acetyl-galactosamine; MTP, microsomal triglyceride transfer protein; TLC, thin layer chromatography;

Unless specified to the contrary, it is intended that every maximum numerical limitation given throughout this description includes every lower numerical limitation, as if such lower numerical limitations were expressly written herein. Every minimum numerical limitation given throughout this specification will include every higher numerical limitation, as if such higher numerical limitations were expressly written herein. Every numerical range given throughout this specification will include every narrower numerical range that falls within such broader numerical range, as if such narrower numerical ranges were all expressly written herein.

The disclosure includes all polynucleotide sequences described herein expressly and by reference, and every polynucleotide sequence referred to herein includes its complementary sequence, and its reverse complement. All segments of polynucleotides from 10 nucleotides to the entire length of the polynucleotides, inclusive, and including numbers and ranges of numbers there between are included. All nucleotide sequences associated with any database accession numbers are incorporated herein by reference as they exist in the database as of the date of the filing of this application or patent. The disclosure includes all polynucleotide sequences described herein expressly or by reference that are between 80.0% and 99.9% identical to the described sequences.

As used in the specification and the appended claims, the singular forms “a” “and” and “the” include plural referents unless the context clearly dictates otherwise. Ranges may be expressed herein as from “about” one particular value, and/or to “about” another particular value. When such a range is expressed, another embodiment includes from the one particular value and/or to the other particular value. Similarly, when values are expressed as approximations, by the use of the antecedent “about” or “approximately” it will be understood that the particular value forms another embodiment. The term “about” and “approximately” in relation to a numerical value encompass variations of +/−10%, +/−5%, or +/−1%.

In embodiments, a therapeutically effective amount of a described double stranded RNA complex in is administered to an individual in need thereof.

A double stranded RNA complex includes two strands that anneal to one another under stringent or semi-stringent conditions. The strand that is optionally not modified may be fully complementary to the modified strand, or double stranded RNA complex may comprise mismatches between the strands.

In embodiments, the double stranded polynucleotide complex comprises one strand which optionally comprises no modifications, and a second strand comprising modifications and can form the double stranded polynucleotide complex with the first strand. The second strand comprises a sequence selected from the group sequences consisting of the following sequences:

| | | | | SEQ ID |
|---|---|---|---|---|
| | | | | NO: |
| miR30c-C1 | C1 | miR-30c-1-3p | (pC)•U•gGgAgAgGgUuGuUuAcUc•C | 1 |
| miR30c-C2 | C2 | miR-30c-1-3p | (pC)•UgGgAgAgGgUuGuUuAc•U•c•C | 2 |
| miR30c-C3 | C3 | miR-30c-1-3p | (pc)•U•gGgAgAgGgUuGuUuAc•U•c•C | 3 |
| miR30c-C4 | C4 | miR-30c-1-3p | GgGaGaGgGuUgUuUaCuC(pC)•u | 4 |
| miR30c-C5 | C5 | miR-30c-1-3p | GgGaGaGgGuUgUuUaCu•C•(pC)•u | 5 |
| miR30c-C6 | C6 | miR-30c-1-3p | C•u•GgGaGaGgGuUgUuUaCuC(pC)u | 6 |

wherein upper case letters signify a 2′-deoxy-2′-fluoro (2′-F) ribosugar modification; lower case letters signify a 2′-O-methyl (2′-OMe) ribosugar modification; pC signifies 2′-a GalNAc clicked cytidine, and the symbol signifies a phosphorothioate linkage. A modified strand is referred to herein from time to time using the C1, C2, C3, C5, C6 and C6 designations as described above.

In embodiments, one or both strands in the described RNA complex may comprise additional modifications. In embodiments, such modifications include, in addition to the modifications described above, modified ribonucleotides or deoxyribonucleotide, and thus include RNA/DNA hybrids. In non-limiting examples, modified ribonucleotides may comprise methylations and/or substitutions of the 2′ position of the ribose moiety with an —O— alkyl group containing 1-6 saturated or unsaturated carbon atoms, or with an —O-aryl group having 3-6 carbon atoms, wherein such alkyl or aryl group may be unsubstituted or may be substituted, e.g., with halo, hydroxy, trifluoromethyl, cyano, nitro, acyl, acyloxy, alkoxy, carboxyl, carbalkoxyl, or amino groups; or with a hydroxy, an amino or a halo group. In embodiments modified nucleotides comprise methyl-cytidine and/or pseudo-uridine. The nucleotides may be linked by phosphodiester linkages or by a synthetic linkage, i.e., a linkage other than a phosphodiester linkage. Examples of inter-nucleoside linkages in the polynucleotide agents that can be used in the disclosure include, but are not limited to, phosphodiester, alkylphosphonate, phosphorothioate, phosphorodithioate, phosphate ester, alkylphosphonothioate, phosphoramidate, carbamate, carbonate, morpholino, phosphate triester, acetamidate, carboxymethyl ester, or combinations thereof

The term “therapeutically effective amount” as used herein refers to an amount of a described RNA complex to achieve, in a single dose or multiple doses, the intended purpose of treatment. The amount desired or required will vary depending its mode of administration, patient specifics and the like. Appropriate effective amounts can be determined by one of ordinary skill in the art informed by the instant disclosure using routine experimentation. In embodiments, a therapeutically effective amount of a described RNA complex is administered to an individual in need thereof. In embodiments, a described RNA complex is administered to an individual in an expression vector and viral vector free manner, i.e., the RNA complex is administered as an RNA complex without expression of the RNA within a cell and without any lipids. In embodiments, a described RNA complex alone is administered to an individual. In embodiments, a composition wherein a described RNA complex is the only RNA complex in the composition is administered.

In embodiments, an agent described herein is administered as a double stranded polynucleotide complex without a liposomal component, and without an expression vector that encodes either stand of the complex, such expression vectors including but not limited to viral vectors.

In embodiments, introducing a described double stranded polynucleotide complex into cells inhibits and/or prevents apoB secretion from cells. In embodiments, introducing a described double stranded polynucleotide complex into cells does not reduce apoA1 secretion, or reduces apoA1 secretion less than a control value obtained from introducing into cells a double stranded polynucleotide complex that does not contain a modified strand, or contains different modifications than those described herein.

In embodiments, the cells into which the described double stranded polynucleotide complex is introduced comprise human liver cells, which may include hepatoma cells. In embodiments, the cells are within a human individual. In embodiments, the human individual needs treatment or prophylaxis of a cardiovascular and/or circulatory disorder, such as atherosclerosis. In embodiments, the individual is in need of plasma lipid reduction. In embodiments, the individual has homozygous familial hypercholesterolemia or low density lipoprotein receptor null mutations.

The following Examples are intended to illustrate but not limit the disclosure.

### EXAMPLES

Modified miR-30c Duplexes have Increased Thermal Stability and are Physiologically Active

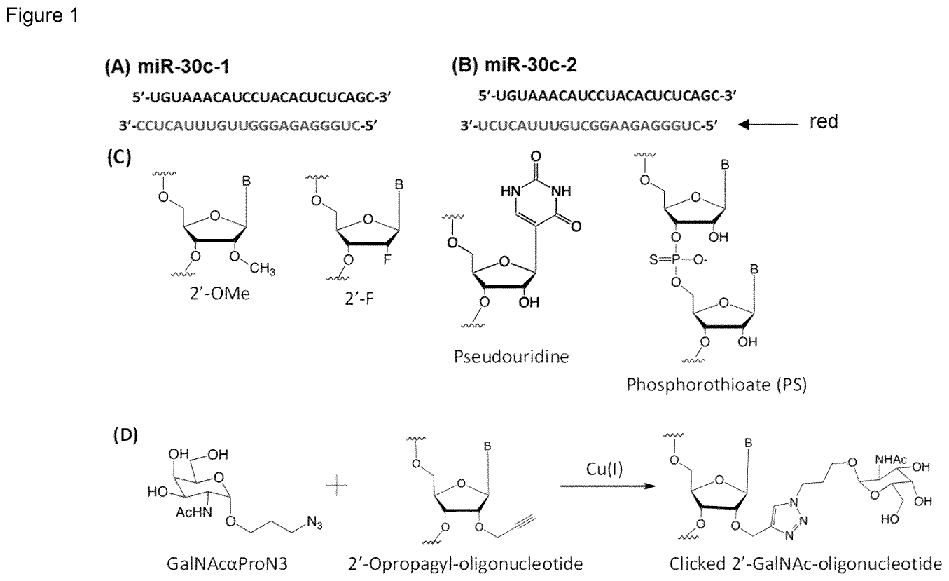

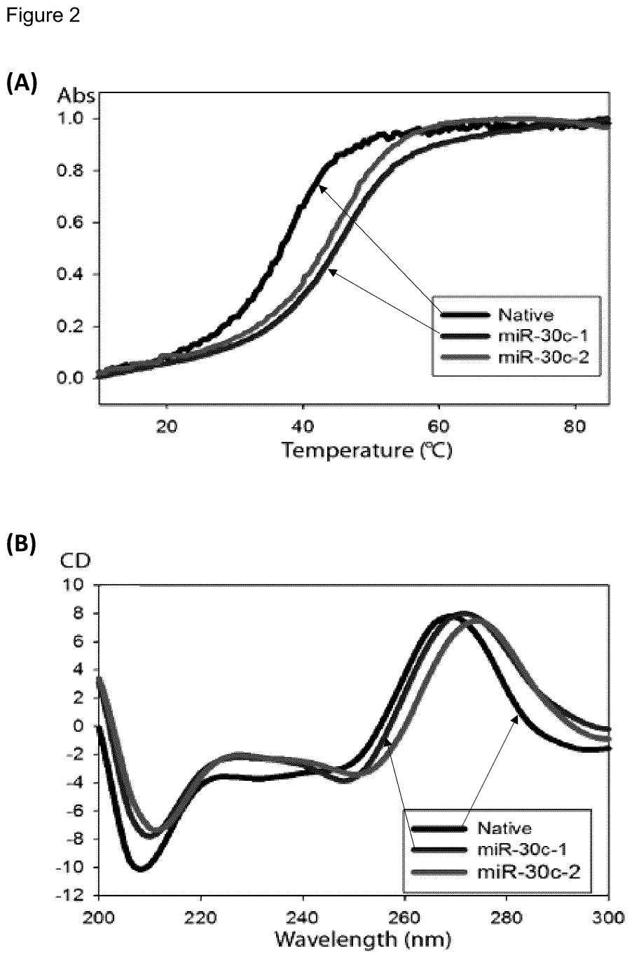

RNA interference-based therapy is becoming a feasible and attractive approach for treating life-threatening diseases that are not easily treatable through conventional small drug molecules. The success of small interfering RNA (siRNA)-based therapy has been due to improvements in the stability, potency, specificity, delivery, and safety of modified siRNAs (30-32). Building on prior advances made in siRNA delivery technology, we synthesized novel analogs of miR-30c to assess whether these approaches might be extended to miRs. Strategically, we decided to modify only the antisense strand (passenger strands, miR-30c-1-3p and miR-30c-2-3p) while leaving the active sense strand (guide strand, miR-30c-5p) untouched to avoid hindering its ability to interact with the RNA-induced silencing complex and recognition of target mRNAs. As shown in FIG. 1A-B, two versions of double stranded mature miR-30c sequences (miR-30c-1 and miR-30c-2) are derived from the products of two independent genes, MIRC1 and MIRC2, contain an identical guide strand (sense, miR-30c-5p, 5′-UGUAAACAUCCUACACUCUCAGC-3′ (SEQ ID NO:7)) but slightly different passenger strands (antisense strands, red, miR-30c-1-3p, 5′-CUGGGAGAGGGUUGUUUACUCC-3′ (SEQ ID NO:8) and miR-30c-2-3p, black, 5′-CUGGGAGAAGGCUGUUUACUCU-3′ (SEQ ID NO:9)). First, we synthesized modified miR-30c-1-3p and miR-30c-2-3p passenger strands by using all 2′-OMe nucleosides (FIG. 1C), then annealed these antisense strands with a sense unmodified or native miR-30c-5p strand to study the biophysical properties of the annealed duplexes. For a control, we annealed unmodified miR-30c-1-3p with native miR-30c-5p. Duplexes of miR-30c-5p with native miR-30c-3p, modified miR-30c-1-3p, or modified miR-30c-2-3p showed similar denaturation curves (FIG. 2A). However, the overall thermal stability of duplexes with modified miR-30c-3p strands increased by ˜7° C., thus indicating enhanced stability of the duplexes. The circular dichroism (CD) spectra showed similar conformations for both native and modified duplexes. These duplexes had a strong positive peak in the range of 260-270 nm (FIG. 2B). Thus, the annealing of miR-30c anti-sense strand containing modified base pairs does not affect its interaction with the miR-30c sense strand in duplex formation and the double helix conformation.

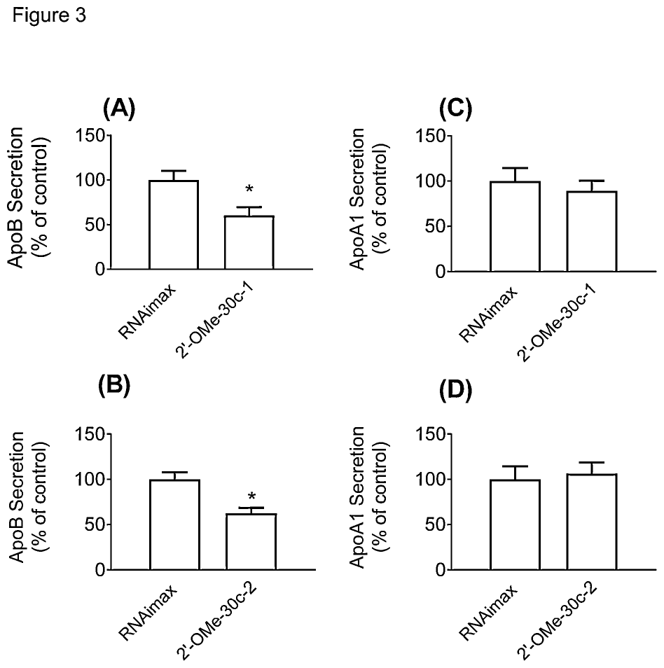

These data indicated that the synthesis of miR-30c-3p with modified nucleotides does not affect either the ability of these modified passenger strands to bind the sense strand or the stability of the dsRNA. Therefore, we evaluated the efficacy of theses miRs in reducing apoB secretion in Huh-7 human hepatoma cells (FIG. 3) by adding them to the media and analyzing their effects on apoB secretion. The duplexes had no effect on apoB secretion when added to cells without transfection reagent. These negative results might have been because the duplexes failed to be delivered to the cells or were not physiologically active. To determine the reasons for their inactivity, we introduced native miR-30c-5p complexed with 2″-OMe modified miR-30c-1-3p (2″-OMe-30C-1) or miR-30c-2-3p (2″-OMe-30c-2) analogs into cells with Lipofectamine RNAiMax reagent. Both modified complexes significantly reduced apoB secretion (FIG. 3A, 3B) without affecting apoA1 secretion (FIG. 3C, 3D), thus indicating that the modified miRs were physiologically active when introduced as lipid complexes. The inability of these complexes to reduce apoB secretion in the absence of lipid-mediated delivery was probably because the RNAs were unable to enter the cells. These data provided crucial preliminary evidence that miR-30c-3p modifications are tolerable if delivered to cells and suggested the feasibility of synthesis of more potent miR-30c analogs by modifying the antisense strands of miR-30c.

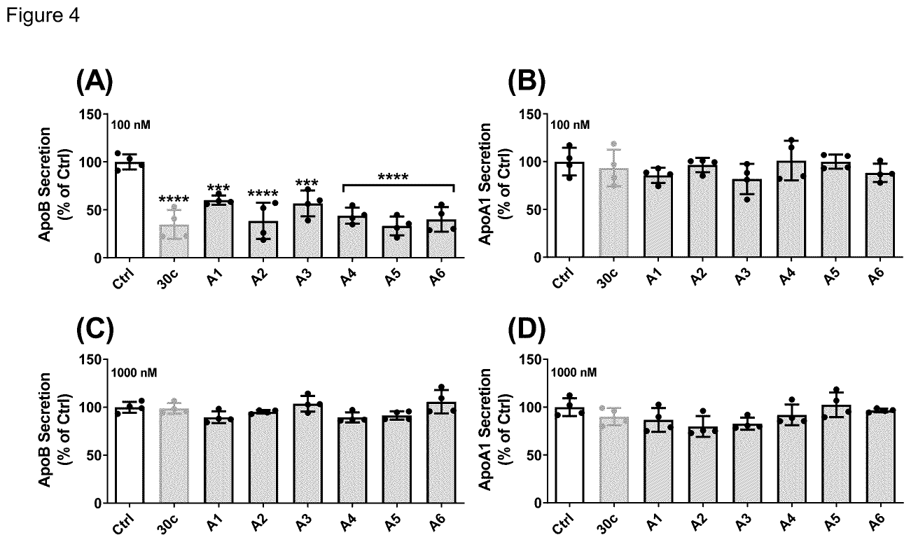

Next, we introduced pseudouridine (ψ) (FIG. 1C) in place of the natural uracil and synthesized six different series A (A1-A6) strands (Table 1). Pseudouridine stabilizes the tertiary structure of tRNA, and the synthetic replacement of all uracil with pseudouridine renders mRNA non-immunogenic and increases its stability (33,34). All analogs were transfected into Huh-7 cells using Lipofectamine RNAiMax reagent (FIG. 4A). For the positive control, we used commercial native miR-30c. For the negative control, we used an unrelated commercially available miR (Ctrl). Compared with the Ctrl, miR-30c and all A-series analogs significantly decreased apoB secretion (FIG. 4A), but apoA1 secretion was unaffected (FIG. 4B). These studies indicated that when delivered to cells, these new synthetic analogs were able to diminish apoB secretion similarly to miR-30c, as compared with Ctrl miR. Next, we asked whether any of these analogs might affect apoB secretion without lipid emulsions. For this purpose, we provided higher concentrations of analogs to cells without Lipofectamine RNAiMax. These analogs and native miR-30c had no effect on apoB and apoA1 secretion (FIGS. 4C and 4D). These studies indicated that these analogs were unable to penetrate cells on their own to reduce apoB secretion; however, they were able to reduce apoB secretion when introduced into cells with Lipofectamine RNAiMax.

MiR-30c-3p Analogs Modified with N-Acetyl-Galactosamine Reduce apoB Secretion without the Use of Lipid Emulsions

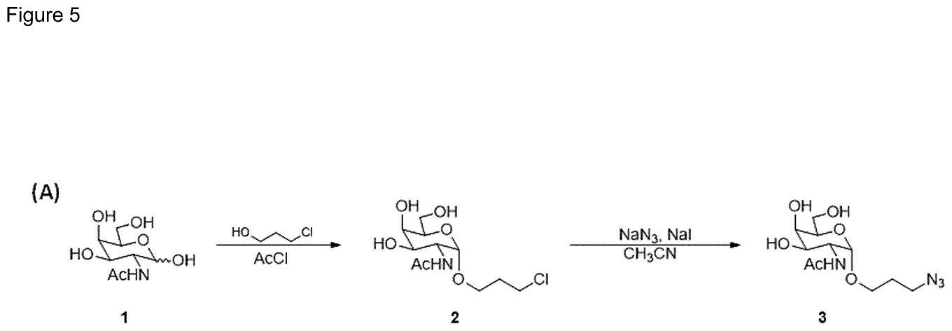

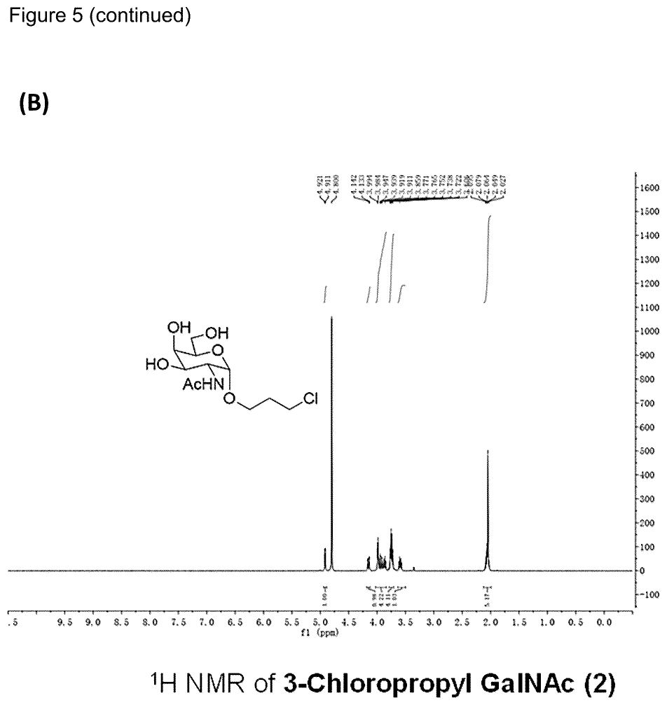

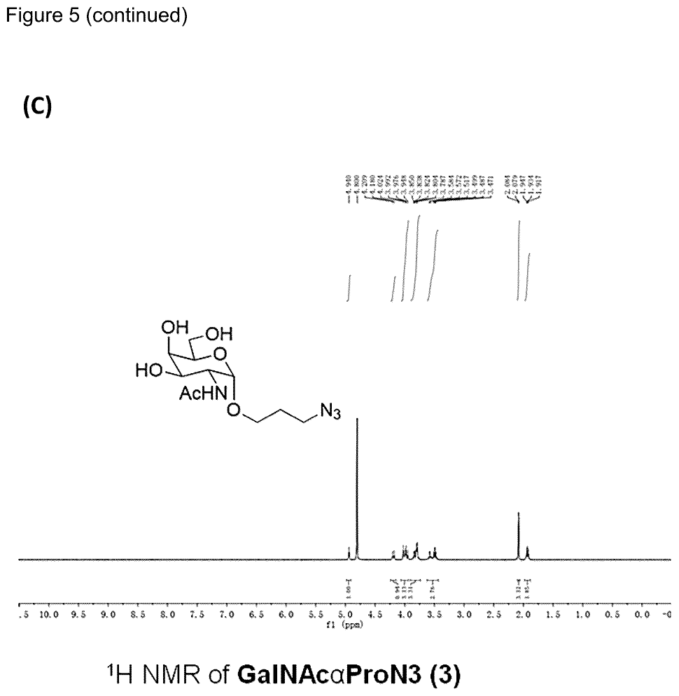

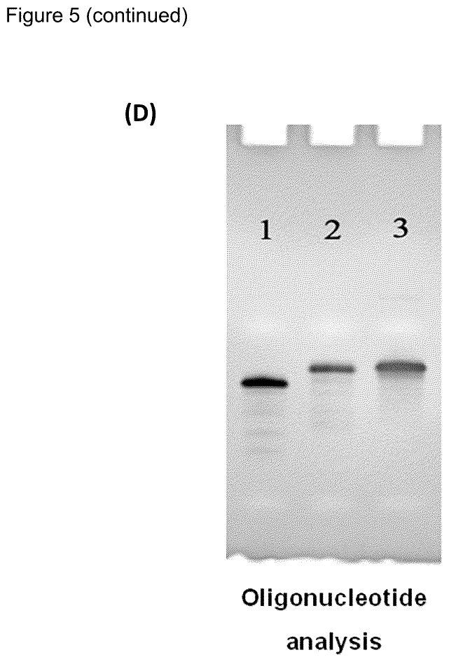

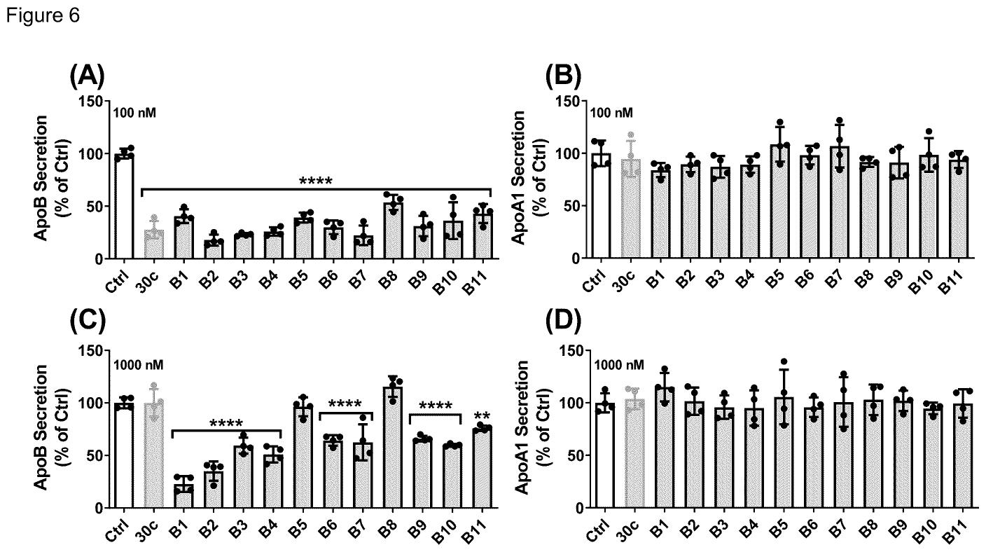

In recent years, the asialoglycoprotein receptor ligand N-acetyl-galactosamine (GalNAc) has been used to deliver anti-sense and siRNA oligonucleotides to liver cells (35-41). Therefore, we introduced one GalNAc modified nucleotide at both ends as well as more than one GalNAc modified residue in the antisense strands. First, we synthesized 3-chloropropyl GalNAc (FIG. 5A, 2) and 3-azidopropyl 2-acetamido-2-deoxy-α-D-galactopyranoside (GalNAcαProN3; FIG. 5A, 3). Their correct synthesis was confirmed by ¹H NMR spectra (FIG. 5B, 5C) and 15% polyacrylamide analytical 8 M urea gel electrophoresis (FIG. 5D). Second, the GalNAcProN3 was attached to 2′-(O-propargyl)-cytosine through click chemistry (42,43). Third, GalNAc-cytidine (pC) was used during the synthesis of miR-30c-3p strands (Table 2). During these syntheses, we also incorporated (2′-deoxy-2′-fluoro ribosugar modified nucleosides to increase the biological stability (44) of these RNA strands (Table 2). Fourth, these analogs were transfected into cells with Lipofectamine RNAiMax. All analogs potently inhibited apoB secretion in Huh-7 cells (FIG. 6A) and therefore were biologically active. ApoA1 secretion was unaffected by these analogs (FIG. 6B). Fifth, we evaluated the efficacy of these series B analogs in reducing apoB secretion when provided to cells without Lipofectamine RNAiMax. Similarly to miR-30c, compounds B5 and B8 had no effect on apoB secretion with respect to the Ctrl (FIG. 6C). However, all other analogs reduced apoB secretion from 40% to 80%. None of these analogs affected apoA1 secretion (FIG. 6D). The most potent analogs inhibiting apoB secretion were B1 and B2 (FIG. 6C). Both these analogs contain one copy of GalNAc at either the 5′- or 3′-end. Introduction of multiple GalNAc modified nucleotides, as in B5 and B8, resulted in a loss of biological activity. Thus, analogs with one GalNAc at either end are suitable for cellular delivery without liposomes.

The GalNAc Modified miR-30c Analogs Significantly Reduce MTP Activity

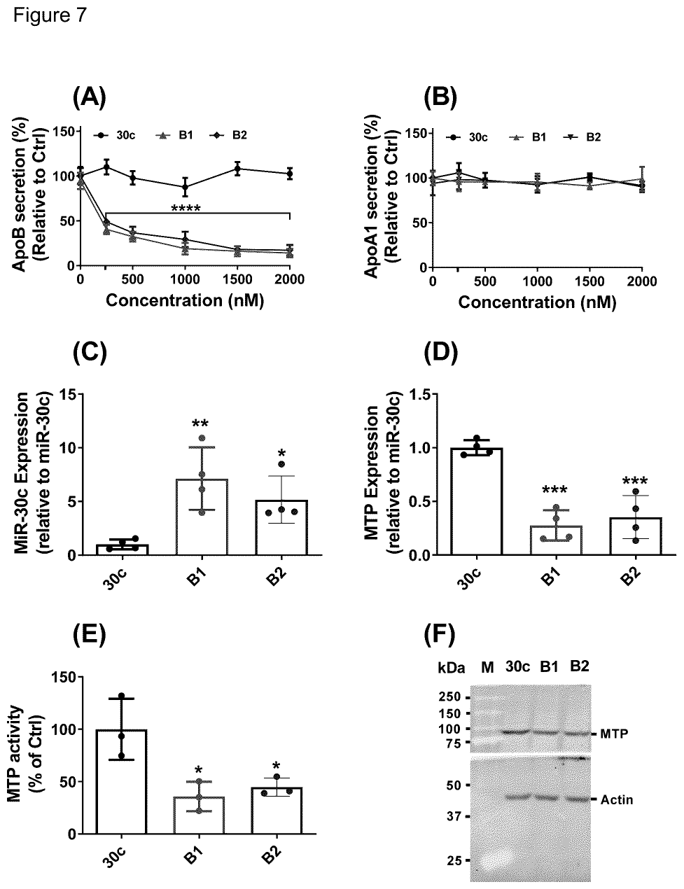

Because analogs B1 and B2 potently inhibited apoB secretion, we performed concentration dependent studies to assess their potency. Both analogs showed a concentration dependent decrease in apoB secretion with an IC50 of 250 nM, but miR-30c had no effect (FIG. 7A). Under similar conditions, different concentrations of miR-30c, B1, and B2 had no effect on apoA1 secretion (FIG. 7B). Next, we exposed cells to 250 nM of B1 and B2 to assess the delivery of miR-30c to cells and its effects on MTP. The levels of miR-30c compared with control miR-30c transcripts were significantly higher (˜7-fold) in the B1-exposed cells and (˜5-fold) in the B2-exposed cells (FIG. 7C). We previously showed that miR-30c diminishes apoB secretion by reducing MTP activity (23-25,28,29). Therefore, we asked whether the analogs B1 and B2 might affect MTP transcripts, protein and activity. Both analogs B1 and B2, compared to miR-30c, reduced MTP mRNA and activity by more than 50% (FIG. 7D, 7E). Western blot analysis also showed a similar decrease in MTP protein (FIG. 7F). These studies suggest that analogs B1 and B2 are likely to reduce apoB secretion in Huh-7 cells by lowering MTP expression similarly to native miR-30c.

Modified miR-30c Analogs with GalNAc and Phosphorothioate at the 5′- or 3′-End have Elevated Potency

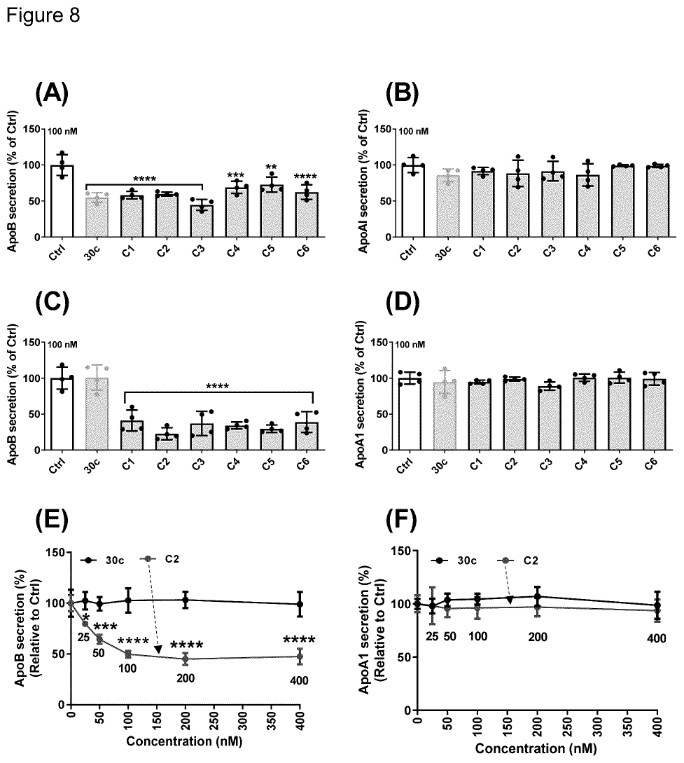

In FIG. 7A, a significant reduction in apoB secretion was observed with 250 nM of analogs B1 and B2. Because our long-term goal is to develop analogs as potential therapeutic drugs, we focused on producing analogs that are more efficacious. Recent studies have suggested that the placement of phosphorothioate linkages improves both the specificity and silencing activity of siRNAs (37,40). Therefore, we synthesized new series C analogs of compounds B1 and B2 by using phosphorothioate linkages (Table 3). These analogs were transfected into cells with Lipofectamine RNAiMax and were found to inhibit apoB secretion (FIG. 8A) without affecting apoA1 secretion (FIG. 8B) in Huh-7 cells, thus suggesting that they are physiologically active. Next, we evaluated their efficacy when they were provided to cells without Lipofectamine RNAiMax. All analogs potently inhibited apoB secretion (FIG. 8C), but had no significant effect on apoA1 secretion (FIG. 8D). In all cases, >50% inhibition of apoB secretion was seen with 100 nM concentrations of these analogs (FIG. 8C). A comparison of the data in FIG. 8A and FIG. 8C suggested that these analogs might be more potent when delivered without lipid emulsions. Next, we verified the effects of different concentrations of the most potent analog, C2, on apoB secretion in Huh-7 cells (FIG. 8E). The analog C2 yielded a concentration dependent decrease in apoB secretion, with an IC50 value of 20 nM. At higher concentrations, the maximum reduction in apoB secretion was ˜60% (FIG. 8E). At all concentrations, analog C2 had no effect on apoA1 secretion (FIG. 8F). These studies indicated that the addition of phosphorothioate linkages increases the efficacy of different analogs in decreasing apoB secretion and that analog C2 is a potent inhibitor of apoB secretion.

### Discussion

Previous work has shown that miR-30c is a good candidate to lower plasma lipids without causing hepatosteatosis, but requires additional agents. This disclosure describes development of potent miR-30c analogs that are expected to be useful as therapeutic agents for lowering plasma lipid levels, among other uses as described herein. The successful synthesis of an azido-modified GalNAc moiety (GalNAcαProN3) allowed us to selectively attach the GalNAc molecule at any location of the RNA oligonucleotide modified with an alkyne group. This synthesis design provided the flexibility to explore the most effective modified passenger strand of miR-30c with the best cell-penetrating capacity. This design could also be extended to other miR systems. By introducing a variety of chemical modifications and phosphorothioate linkages, we improved the efficacy of the miR-30c analogs. Addition of GalNAc residues at either end circumvented the need for lipid emulsions for delivery to hepatoma cells. Mechanistic studies showed that these analogs reduced MTP mRNA and thus behaved similarly to native miR-30c. Furthermore, these analogs had no effect on apoA1 secretion; therefore, the decreased apoB was a specific response to these analogs and was not secondary to cellular toxicity. These studies provide evidence that the passenger strand of miR-30c can be modified to enhance cellular delivery without lipid emulsions. This system therefore has certain advantages relative to delivery with lipid emulsions.

In summary, the disclosure demonstrates successful synthesis of various miR-30c analogs that potently inhibit MTP activity and apoB secretion. The disclosure demonstrates that modification of miR-30c in the passenger strand can improve its stability and delivery into hepatoma cells.

### EXPERIMENTAL PROCEDURES

Synthesis of Chemically Modified Novel miR-30c-3p Analogs

Anhydrous solvents were used and redistilled through standard procedures. All solid reagents were dried under a high vacuum line before use. Air sensitive reactions were performed under argon. Analytical thin layer chromatography (TLC) plates pre-coated with silica gel F254 (Aldrich, #717185) were used for monitoring reactions. The ¹H NMR spectra were measured on a Brucker Ascend 500 MHz spectrometer. Chemical shift values are reported in ppm. High-resolution mass spectroscopy was achieved with a quadrupole time of flight spectrometer at the University at Albany, SUNY.

Native RNAs were custom synthesized by Integrated DNA Technologies. We used an automated RNA/DNA synthesizer (ASM800, BIOSSET Ltd.) to synthesize modified antisense oligonucleotides at 1 micromole scale with commercial phosphoramidites and reagents from Chemgenes. The modified and native phosphoramidites were dissolved in anhydrous acetonitrile to obtain a concentration of 0.07 M. Trichloroacetic acid (3%) in dichloromethane was used for detritylation, and the coupling step was performed with 5-ethylthio-1H-tetrazole (0.25 M in acetonitrile) for 12 min. The unreacted 5′-OH was capped with CapA solution (80% tetrahydrofuran/10% acetic anhydride/10% 2,6-Lutidine) and CapB solution (16% N-methyl imidazole in tetrahydrofuran). Oxidation on the phosphate backbone was performed with 20 mM iodine solution in pyridine/tetrahydrofuran/water. Phosphorothioate backbones were synthesized with sulfurization reagent (3-((dimethylamino-methylidene)amino)-3H-1,2,4-dithiazole-3-thione, instead of the iodine solution in the oxidation step. All the reagents were of oligo-synthesizer grade from Chemgenes.

The synthesis of modified miR-30c passenger strands was performed with the dimethoxyltrityl-off mode of the RNA/DNA synthesizer. The protection on bases was removed, and the oligonucleotides were cleaved from the solid support with concentrated aqueous ammonium hydroxide at room temperature for 18 hours. The resulting RNA solution was dried with a speed vacuum concentrator, and the pellet was redissolved with 100 μL dimethylsulfoxide and incubated with 125 μL of triethylamine trihydrofluoride (Aldrich, #344648) at 65° C. for 2.5 hours. The RNA was precipitated by addition of 25 μL of 3 M sodium acetate solution and 1 mL ethanol and subsequent cooling of the mixture at −80° C. for 3 hours before centrifugation. The RNAs were dissolved in RNase free water and desalted again prior to storage at −20° C. in RNase free water. The purity of modified-RNA samples was confirmed by analytical denaturing gel electrophoresis (15% polyacrylamide with 8 M urea) and electrospray ionization mass spectrometry.

Annealing of Modified miR-30c-3p Strands with Native miR-30c-5p Strand and UV Thermal Denaturation Studies

The modified miR-30c-3p RNAs were annealed with native miR-30c-5p in sodium phosphate buffer (10 mM, pH 6.5) containing 100 mM NaCl. The solutions were heated at 95° C. for 3 min, then cooled to room temperature at a rate of 1° C./min, and stored overnight at 4° C. before use. For thermal denaturation studies, data points were acquired at 260 nm by heating and cooling from 5° C. to 85° C. (two cycles, four ramps) at a rate of 0.5° C./min, with a Cary-300 UV-visible spectrometer equipped with a temperature controller system. The thermodynamic parameters of each duplex strand were obtained by fitting the melting curves in Meltwin software (45).

### Circular Dichroism Spectroscopy

Circular dichroism spectra were recorded at room temperature on a JASCO-815 spectropolarimeter over a wavelength range of 200-300 nm with a 1 cm path length quartz cuvette with a scanning speed of 100 nm/min, bandwidth of 1.0 nm, and digital integration time of 1.0 s. Each spectrum was averaged from four scans and baseline-corrected against the buffer.

### Synthesis of GalNAcαProN3, 3

Published protocols (46) were followed to synthesize 3-chloropropyl GalNAc 2 and GalNAcαProN3 3.

### Synthesis of 3-Chloropropyl GalNAc 2.

To a solution of N-acetyl-D-galactosamine (330 mg, 1.5 mmol) in 3-chloropropanol (5 mL), acetyl chloride (0.13 mL, 1.8 mmol) was added at 0° C. The reaction mixture was heated at 70° C. for 15 h. The solution was concentrated, and the residue was purified by silica gel chromatography, thus yielding 3-chloropropyl GalNAc 2 (200 mg, 45%) as a white solid. TLC R_{f}=0.5 (20% MeOH in CH₂Cl₂). ¹H NMR (500 MHz, D₂O) δ 4.92 (d, J=4.0 Hz, 1H), 4.15 (dd, J=4.4, 12.8 Hz, 1H), 4.00-3.84 (m, 4H), 3.77-3.72 (m, 4H), 3.62-3.56 (m, 1H), 2.10-2.02 (m, 5H).

GalNAcαProN3 3. 3-chloropropyl GalNAc 2 (200 mg, 0.671 mmol) was dissolved in CH₃CN (6 mL) by heating the solution. NaN₃ (436 mg, 6.71 mmol) and NaI (101 mg, 0.671 mmol) were added. The resulting mixture was stirred at 60° C. for h. The solution was concentrated, and the residue was purified by silica gel chromatography, thus yielding GalNAcαProN3 3 (110 mg, 54%) as a white solid. TLC R_{f}=0.4 (20% MeOH in CH₂Cl₂). ¹H NMR (500 MHz, D₂O) δ 4.94 (m, 1H), 4.21-4.18 (m, 1H), 4.03-3.94 (m, 3H), 3.85-3.78 (m, 3H), 3.59-3.47 (m, 3H), 2.08 (d, 3H), 1.95-1.91 (m, 2H).

### Synthesis of 2′-GalNAc-Modified RNA Strands

The propargyl modified RNA oligonucleotides were first synthesized according to the solid phase synthesis procedure with commercially available 2′-(O-propargyl)-phosphoramidite building blocks from Chemgenes. GalNAc modified RNA strands were produced through mixture of the propargyl-RNA (1 equivalent) with 100 equivalents of azido-modified GalNAc (GalNAcαProN3) in a 1.5 mL microcentrifuge tube. In a separate tube, 22 equivalents of copper(I) bromide (CuBr) (100 mM in 25% tBuOH/75% dimethylsulfoxide) and 20% acetonitrile were mixed and transferred to the RNA solution. The mixture was shaken at room temperature for 12 h. The RNA was then precipitated with 3 M sodium acetate and ethanol after storage at −80° C. for 3 hours. The RNA was pelleted by centrifugation at 14,000 rpm (Eppendorf 5424) for 15 minutes. The RNA pellet was resuspended in 500 μL RNase-free water, and the solution was further desalted with SepPak C18 cartridges (Waters™). The elution fractions with RNA were combined and concentrated with an oligonucleotide concentrator speed vacuum. The resulting click reaction products were monitored with analytical gel electrophoresis with 15% polyacrylamide containing 8 M urea.

### Cell Culture Studies

The Huh-7 cells were maintained in Dulbecco's modified Eagle's medium (DMEM) containing 10% fetal bovine serum (FBS) and 1% L-glutamine in 75 cm 2 culture flasks with vent caps (Corning®, #430641U) at 37° C. and 5% CO₂ in a humidified incubator. Two types of studies were performed to assess the ability of different analogs to decrease apoB secretion. First, analogs were introduced into cells with Lipofectamine RNAiMax transfection reagent. Second, cells were exposed to different analogs without the use of any transfection reagent. In both of these experiments, Huh-7 cells were seeded at a concentration of 100,000 cells/well in a six-well plate in 2 mL of the aforementioned medium. The next day, 1 mL of fresh Opti-MEM™ I reduced serum medium (Gibco) (for transfection experiments) or DMEM containing 10% FBS (for non-liposome mediated transfection) was added to the cells. The Huh-7 cells in the Opti-MEM™ I reduced serum medium were transfected with commercially available non-specific control miR (Ctrl), miR-30c mimic (positive control), or our experimental novel synthetic miR-30c analogs with Lipofectamine RNAiMax reagent (Invitrogen) according to the manufacturer's protocol. Briefly, each miR was mixed with RNAiMax at a ratio of 3:1 and incubated for 30 minutes at room temperature. This mixture was then added to cells. In experiments testing liposome independent delivery of miR analogs, cells in DMEM (10% FBS) were exposed to these analogs without Lipofectamine RNAiMax reagent. On day 2, in both cases, 1 mL of fresh DMEM (10% FBS) was added to the cells. At 72 h after the start of transfection, the media were changed, and 1 mL of fresh DMEM (10% FBS) was added to the cells. After overnight incubation, the media were collected for apoB and apoA1 measurements. Cells were washed and collected in the presence of protease inhibitor cocktail (Sigma-Aldrich, #P2714) for protein estimation and determination of the activity of MTP as previously described (47).

ApoB and apoA1 Measurements

The apoB levels in the collected media were determined with a human apoB ELISA development kit (MABTECH Inc, #3715-1H-6) in 96-well ELISA plates (Thermo Fisher Scientific, #07-200-640) with 3,3′,5,5′ tetramethylbenzidine substrate (Thermo Fisher Scientific, #4041). The apoB concentration was calculated with apoB standard provided by the manufacturer in parallel in the same plate. The medium apoB values were normalized to the total protein in the respective wells. Total cellular protein concentrations were quantified with a Coomassie (Bradford) protein assay kit (Thermo Fisher Scientific, #23200). The apoB concentrations in the media of control miR (Ctrl)-transfected cells were set at 100%. ApoB secretion by cells exposed to miR-30c mimic or newly synthesized miR-30c analogs is presented as a percentage of this value.

The apoA1 levels in the collected media were determined with a human apolipoprotein A-I/ApoA1 DuoSet ELISA kit (R&D Systems, #DY3664) in 96-well ELISA plates (Thermo Fisher Scientific, #7-200-640). Substrate (#DY999) and stop (#DY994) solutions were from R&D Systems. The apoA1 concentration was calculated with an apoA1 standard curve prepared in parallel with standards provided by the manufacturer (R&D Systems, #DY3664). ApoA1 levels in the media were normalized to total protein in the respective wells. The concentrations of apoA1 in the control miR (Ctrl)-transfected cells were set to 100%, and those in miR-30c mimic treated cells were normalized to this value.

Measurement of miR-30c and MTP Transcript Levels

For miR-30c quantification by quantitative RT-PCR, cDNA was synthesized from RNA isolated from cells with a TaqMan MicroRNA Reverse Transcription kit (Applied Biosystems, #4366597). The miR-30c and U6-specific primers were purchased from Thermo Fisher Scientific. For quantitative RT-PCR, TaqMan™ Universal Master Mix II (Applied Biosystems, #4440043) was used. For miR-30c quantification, the Ct method with normalization to U6 was used, and the data are presented as fold changes.

For MTP quantification by quantitative RT-PCR, cDNA was synthesized from isolated RNA with a High-Capacity cDNA Reverse Transcription Kit (Thermo Fisher Scientific, #4368813). The human MTP and β-actin-specific primers were purchased from Integrated DNA Technologies (MTP primers, 5′-TGTGGCCTTACTATGGAGGAA-3′ (SEQ ID NO:32) and 5′-AAGGAGCGTAGGTCTTTGCAG-3′ (SEQ ID NO:10); β-actin primers, 5′-AGAGCTACGAGCTGCCTGAC-3′ (SEQ ID NO:11) and 5′-AGCACTGTGTTGGCGTACAG-3′ (SEQ ID NO:12)). For quantitative RT-PCR, PowerTrack SYBR Green Master Mix (Thermo Fisher Scientific, #A46109) was used. For MTP quantification, the Ct method with normalization to β-actin was used, and data are presented as fold change with respect to controls.

### MTP Protein Levels and Activity Measurements

Transfected Huh-7 cells were washed with ice-cold phosphate buffered saline and scraped from the wells in ice cold buffer K (1 mM Tris-HCl, 1 mM EGTA and 1 mM MgCl₂, pH 7.6) containing protease inhibitor cocktail (Sigma-Aldrich, #P2714). Cells were manually lysed through 20 passes through a BD PrecisionGlide™ 25G needle, and the total protein concentrations were measured with a Coomassie (Bradford) protein assay kit (Thermo Fisher Scientific, #23200). Proteins (25 μg) were resolved by SDS-PAGE (10%). A polyclonal rabbit primary antibody to human MTP (Abcam, #ab63467) and a monoclonal rabbit antibody to β-actin (Cell Signaling Technology, #8457) were used at 1:1000 dilution. Anti-rabbit IgG, HRP-linked secondary antibody (Cell Signaling Technology, #7074) was used at 1:2000 dilution. The blots were developed with a ChemiDoc™-Touch Imaging system (Bio-Rad). To determine the MTP activity, 50 μg of total proteins was used. Fluorescently labeled triglyceride transfer assays were performed as previously described (25,28,29,47).

### Statistical Analysis

We performed statistical analysis in GraphPad Prism versions 8 and 9. All data are represented as mean±SD. The symbols *, **, and *** represent significance at p<0.05, p<0.01, and p<0.001, respectively.

### Supplemental Example

### Studies in Human Hepatoma Huh-7 Cells:

It is known that ablation of ApoB secretion results in hepatosteatosis. However, we have previously shown that miR-30c reduces apoB secretion without causing steatosis. Data presented before showed that analog C2 reduces ApoB secretion without affecting ApoA1 secretion. Therefore, we investigated whether analog C2 causes steatosis or not. Huh-7 cells were treated with or without analog C2 and media was collected to measure ApoB and ApoA1 levels. Cells were washed with phosphate buffer saline (PBS) and lipids were extracted using isopropanol to measure triglyceride and cholesterol.

### Studies in Human Primary Hepatocytes:

To extend our studies about the efficacy of different miR-30c analogs beyond human hepatoma Huh-7 cells, we purchased human primary hepatocytes (H1000.H15B+; Lot No. HC4-25) from Sekisui XenoTech. Cells were thawed using Sekisui XenoTech's thawing protocol and OptiThaw Hepatocyte Kit. After seeding in collagen-coated plates, cells were treated with the analog C2. After 72 h, media was collected to measure ApoB and apoA1 levels using ELISA.

To determine whether analog C2 increases cellular lipid levels, cells were washed with PBS. To each well, 500 μl of isopropanol was added and plate was incubated overnight at 4° C. Next day the supernatants were collected, dried and resuspended in 100 μl of isopropanol followed by the lipid measurements. Total triglycerides and cholesterol were measured using commercial kits (Pointe Scientific). To each well, 500 μl of 0.1N NaOH was added to determine the protein concentration for normalization.

### REFERENCES—THIS REFERENCE LISTING IS NOT AN INDICATION THAT ANY OF THE REFERENCES ARE MATERIAL TO PATENTABILITY

1. Blumenthal, R. S. (2000) Statins: effective antiatherosclerotic therapy. Am. Heart J 139, 577-583
2. Ehrenstein, M. R., Jury, E. C., and Mauri, C. (2005) Statins for atherosclerosis—as good as it gets? N. Engl. J. Med 352, 73-75
3. LaRosa, J. C., Grundy, S. M., Waters, D. D., Shear, C., Barter, P., Fruchart, J. C., Gotto, A. M., Greten, H., Kastelein, J. J., Shepherd, J., and Wenger, N. K. (2005) Intensive lipid lowering with atorvastatin in patients with stable coronary disease. N. Engl. J. Med 352, 1425-1435
4. Stoekenbroek, R. M., Kastelein, J. J., and Huijgen, R. (2015) PCSK9 inhibition: the way forward in the treatment of dyslipidemia. BMC. Med 13, 258 Voora, D., Shah, S. H., Reed, C. R., Zhai, J., Crosslin, D. R., Messer, C., Salisbury, B. A., and
5. Ginsburg, G. S. (2008) Pharmacogenetic predictors of statin-mediated low-density lipoprotein cholesterol reduction and dose response. Circ. Cardiovasc. Genet 1, 100-106
6. Ahmad, Z. (2014) Statin intolerance. Am. J. Cardiol 113, 1765-1771
7. Saxon, D. R., and Eckel, R. H. (2016) Statin Intolerance: A Literature Review and Management Strategies. Prog. Cardiovasc. Dis 59, 153-164
8. Rader, D. J., and Kastelein, J. J. (2014) Lomitapide and mipomersen: two first-in-class drugs for reducing low-density lipoprotein cholesterol in patients with homozygous familial hypercholesterolemia. Circulation 129, 1022-1032
9. Raal, F. J., Stein, E. A., Dufour, R., Turner, T., Civeira, F., Burgess, L., Langslet, G., Scott, R., Olsson, A. G., Sullivan, D., Hovingh, G. K., Cariou, B., Gouni-Berthold, I., Somaratne, R., Bridges, I., Scott, R., Wasserman, S. M., and Gaudet, D. (2015) PCSK9 inhibition with evolocumab (AMG 145) in heterozygous familial hypercholesterolaemia (RUTHERFORD-2): a randomised, double-blind, placebo-controlled trial. Lancet 385, 331-340
Hussain, M. M., Shi, J., and Dreizen. P. (2003) Microsomal triglyceride transfer protein and its role in apolipoprotein B-lipoprotein assembly. J. Lipid Res 44, 22-32
11. Sirwi, A., and Hussain, M. M. (2018) Lipid transfer proteins in the assembly of apoB-containing lipoproteins. J. Lipid Res 59, 1094-1102
12. Cuchel, M., Meagher, E. A., du Toit, T. H., Blom, D. J., Marais, A. D., Hegele, R. A., Averna, M. R., Sirtori, C. R., Shah, P. K., Gaudet, D., Stefanutti, C., Vigna, G. B., Du Plessis, A. M., Propert, K. J., Sasiela, W. J., Bloedon, L. T., and Rader, D. J. (2013) Efficacy and safety of a microsomal triglyceride transfer protein inhibitor in patients with homozygous familial hypercholesterolaemia: a single-arm, open-label, phase 3 study. Lancet 381, 40-46
13. Samaha, F. F., McKenney, J., Bloedon, L. T., Sasiela, W. J., and Rader, D. J. (2008) Inhibition of microsomal triglyceride transfer protein alone or with ezetimibe in patients with moderate hypercholesterolemia. Nat. Clin. Pract. Cardiovasc. Med 5, 497-505
14. Burnett, J. R., and Watts, G. F. (2007) MTP inhibition as a treatment for dyslipidaemias: time to deliver or empty promises? Expert. Opin. Ther. Targets 11, 181-189
15. Joy, T. R., and Hegele, R. A. (2008) Microsomal triglyceride transfer protein inhibition-friend or foe? Nat. Clin Pract. Cardiovasc. Med 5, 506-508
16. Hussain, M. M., and Bakillah, A. (2008) New approaches to target microsomal triglyceride transfer protein. Curr. Opin. Lipidol 19, 572-578
17. Cuchel, M., and Rader, D. J. (2013) Microsomal transfer protein inhibition in humans. Curr. Opin Lipidol 24, 246-250
18. Rizzo, M., and Wierzbicki, A. S. (2011) New lipid modulating drugs: the role of microsomal transport protein inhibitors. Curr. Pharm. Des 17, 943-949
19. Bartel, D. P. (2009) MicroRNAs: target recognition and regulatory functions. Cell 136, 215-Krol, J., Loedige, I., and Filipowicz, W. (2010) The widespread regulation of microRNA biogenesis, function and decay. Nat. Rev. Genet 11, 597-610
21. Rupaimoole, R., and Slack, F. J. (2017) MicroRNA therapeutics: towards a new era for the management of cancer and other diseases. Nat Rev Drug Discov 16, 203-222
22. van, R. E., and Kauppinen, S. (2014) Development of microRNA therapeutics is coming of age. EMBO Mol. Med 6, 851-864
23. Irani, S., and Hussain, M. M. (2015) Role of microRNA-30c in lipid metabolism, adipogenesis, cardiac remodeling and cancer. Curr. Opin Lipidol 26, 139-146
24. Zhou, L., Irani, S., Sirwi, A., and Hussain, M. M. (2016) MicroRNAs regulating apolipoprotein B-containing lipoprotein production. Biochim. Biophys. Acta 1861, 2062-2068
25. Soh, J., Iqbal, J., Queiroz, J., Fernandez-Hernando, C., and Hussain, M. M. (2013) MicroRNA-30c reduces hyperlipidemia and atherosclerosis by decreasing lipid synthesis and lipoprotein secretion. Nat. Med 19, 892-900
26. Soh, J., and Hussain, M. M. (2013) Supplementary site interactions are critical for the regulation of microsomal triglyceride transfer protein by microRNA-30c. Nutr Metab (Lond) 10, 56
27. Zhou, L., and Hussain, M. M. (2017) Human MicroRNA-548p Decreases Hepatic Apolipoprotein B Secretion and Lipid Synthesis. Arterioscler Thromb Vasc Biol 37, 786-793
28. Irani, S., Pan, X., Peck, B. C. E., Iqbal, J., Sethupathy, P., and Hussain, M. M. (2016) MicroRNA-30c mimic mitigates hypercholesterolemia and atherosclerosis in mice. J. Biol. Chem 291, 18397-18409
29. Irani, S., Iqbal, J., Antoni, W. J., Aijaz, L., and Hussain, M. M. (2018) MicroRNA-30c reduces plasma cholesterol in familial hypercholesterolemic and Type 2 diabetic mouse models. J. Lipid Res 59, 144-154
30. Weiss, W. A., Taylor, S. S., and Shokat, K. M. (2007) Recognizing and exploiting differences between RNAi and small-molecule inhibitors. Nat Chem Biol 3, 739-744
31. Corey, D. R. (2007) Chemical modification: the key to clinical application of RNA interference? J Clin Invest 117, 3615-3622
32. de Fougerolles, A., Vornlocher, H. P., Maraganore, J., and Lieberman, J. (2007) Interfering with disease: a progress report on siRNA-based therapeutics. Nat Rev Drug Discov 6, 443-453
33. Kariko, K., Muramatsu, H., Welsh, F. A., Ludwig, J., Kato, H., Akira, S., and Weissman, D. (2008) Incorporation of pseudouridine into mRNA yields superior nonimmunogenic vector with increased translational capacity and biological stability. Mol Ther 16, 1833-1840
34. Spenkuch, F., Motorin, Y., and Helm, M. (2014) Pseudouridine: still mysterious, but never a fake (uridine)! RNA Biol 11, 1540-1554
35. Akinc, A., Querbes, W., De, S., Qin, J., Frank-Kamenetsky, M., Jayaprakash, K. N., Jayaraman, M., Rajeev, K. G., Cantley, W. L., Dorkin, J. R., Butler, J. S., Qin, L., Racie, T., Sprague, A., Fava, E., Zeigerer, A., Hope, M. J., Zerial, M., Sah, D. W., Fitzgerald, K., Tracy, M. A., Manoharan, M., Koteliansky, V., Fougerolles, A., and Maier, M. A. (2010) Targeted delivery of RNAi therapeutics with endogenous and exogenous ligand-based mechanisms. Mol. Ther 18, 1357-1364
36. Nair, J. K., Willoughby, J. L., Chan, A., Charisse, K., Alam, M. R., Wang, Q., Hoekstra, M., Kandasamy, P., Kel'in, A. V., Milstein, S., Taneja, N., O'Shea, J., Shaikh, S., Zhang, L., van der Sluis, R. J., Jung, M. E., Akinc, A., Hutabarat, R., Kuchimanchi, S., Fitzgerald, K., Zimmermann, T., van Berkel, T. J., Maier, M. A., Rajeev, K. G., and Manoharan, M. (2014) Multivalent N-acetylgalactosamine-conjugated siRNA localizes in hepatocytes and elicits robust RNAi-mediated gene silencing. J. Am. Chem. Soc 136, 16958-16961
37. Juliano, R. L. (2016) The delivery of therapeutic oligonucleotides. Nucleic Acids Res 44, 6518-6548
38. Liebow, A., Li, X., Racie, T., Hettinger, J., Bettencourt, B. R., Najafian, N., Haslett, P., Fitzgerald, K., Holmes, R. P., Erbe, D., Querbes, W., and Knight, J. (2017) An Investigational RNAi Therapeutic Targeting Glycolate Oxidase Reduces Oxalate Production in Models of Primary Hyperoxaluria. J Am Soc Nephrol 28, 494-503
39. Fitzgerald, K., White, S., Borodovsky, A., Bettencourt, B. R., Strahs, A., Clausen, V., Wijngaard, P., Horton, J. D., Taubel, J., Brooks, A., Fernando, C., Kauffman, R. S., Kallend, D., Vaishnaw, A., and Simon, A. (2017) A Highly Durable RNAi Therapeutic Inhibitor of PCSK9. N Engl J Med 376, 41-51
40. Crooke, S. T., Witztum, J. L., Bennett, C. F., and Baker, B. F. (2018) RNA-Targeted Therapeutics. Cell Metab 27, 714-739
41. Zimmermann, T. S., Karsten, V., Chan, A., Chiesa, J., Boyce, M., Bettencourt, B. R., Hutabarat, R., Nochur, S., Vaishnaw, A., and Gollob, J. (2017) Clinical Proof of Concept for a Novel Hepatocyte-Targeting GalNAc-siRNA Conjugate. Mol. Ther 25, 71-78
42. Rostovtsev, V. V., Green, L. G., Fokin, V. V., and Sharpless, K. B. (2002) A stepwise huisgen cycloaddition process: copper(I)-catalyzed regioselective “ligation” of azides and terminal alkynes. Angew Chem Int Ed Engl 41, 2596-2599
43. Tornoe, C. W., Christensen, C., and Meldal, M. (2002) Peptidotriazoles on solid phase: [1,2,3]-triazoles by regiospecific copper(i)-catalyzed 1,3-dipolar cycloadditions of terminal alkynes to azides. J Org Chem 67, 3057-3064
44. Janas, M. M., Zlatev, I., Liu, J., Jiang, Y., Barros, S. A., Sutherland, J. E., Davis, W. P., Liu, J., Brown, C. R., Liu, X., Schlegel, M. K., Blair, L., Zhang, X., Das, B., Tran, C., Aluri, K., Li, J., Agarwal, S., Indrakanti, R., Charisse, K., Nair, J., Matsuda, S., Rajeev, K. G., Zimmermann, T., Sepp-Lorenzino, L., Xu, Y., Akinc, A., Fitzgerald, K., Vaishnaw, A. K., Smith, P. F., Manoharan, M., Jadhav, V., Wu, J. T., and Maier, M. A. (2019) Safety evaluation of 2′-deoxy-2′-fluoro nucleotides in GalNAc-siRNA conjugates. Nucleic Acids Res 47, 3306-3320
45. McDowell, J. A., and Turner, D. H. (1996) Investigation of the structural basis for thermodynamic stabilities of tandem GU mismatches: solution structure of (rGAGGUCUC)2 by two-dimensional NMR and simulated annealing. Biochemistry 35, 14077-14089
46. Yu, H., Chokhawala, H., Karpel, R., Yu, H., Wu, B., Zhang, J., Zhang, Y., Jia, Q., and Chen, X. (2005) A multifunctional Pasteurella multocida sialyltransferase: a powerful tool for the synthesis of sialoside libraries. J Am Chem Soc 127, 17618-17619
47. Athar, H., Iqbal, J., Jiang, X. C., and Hussain, M. M. (2004) A simple, rapid, and sensitive fluorescence assay for microsomal triglyceride transfer protein. J. Lipid Res 45, 764-772

### Tables:

TABLE 1
MIR-30c analogs and sequences. Underlined upper case letters,
native nucleosides; lower case letters, 2′-O-methyl (2'-OMe)
ribosugar modifications; ψ, pseudouridine.

| | Short | | | SEQ ID |
|---|---|---|---|---|
| Compound | form | Strand | Sequence (5′ to 3′) | NO: |
| Ctrl | Ctrl | Sense | UCACAACCUCCUAGAAAGAGUAGA | 13 |
| miR30c | 30c | miR-30c-5p | UGUAAACAUCCUACACUCUCAGC | 14 |
| miR30c-A1 | A1 | miR-30c-1-3p | CugggagaggguuguuuacucC | 15 |
| miR30c-A2 | A2 | miR-30c-1-3p | CψgggagagggwgψψψacψcC | 16 |
| miR30c-A3 | A3 | miR-30c-1-3p | CψgggAgAgggψψgψψψACψCC | 17 |
| miR30c-A4 | A4 | miR-30c-2-3p | CugggagaaggcuguuuacucU | 18 |
| miR30c-A5 | A5 | miR-30c-2-3p | cψgggagaaggcψgψψψacψcU | 19 |
| miR30c-A6 | A6 | miR-30c-2-3p | CψgggAgAAggCψgψψψACψCU | 20 |

TABLE 2
MIR-30c analogs and sequences containing GalNAc modification.
Upper case letters, 2′-deoxy-2′-fluoro (2′-F) ribosugar modified nucleosides;
lower case letters, 2′-O-methyl (2′-OMe) ribosugar modified nucleosides; (pC),
2′-GalNAc clicked cytidines.

| | Short | | | SEQ ID |
|---|---|---|---|---|
| Compound | form | Strand | Sequence (5′ to 3′) | NO: |
| miR30c-B1 | B1 | miR-30c-1-3p | (pC)UgGgAgAgGgUuGuUuAcUcC | 21 |
| miR30c-B2 | B2 | miR-30c-1-3p | CuGgGaGaGgGuUgUuUaCuC(pC)u | 22 |
| miR30c-B3 | B3 | miR-30c-1-3p | (pC)uGgGaGaGgGuUgUuUaCuC(pC)u | 23 |
| miR30c-B4 | B4 | miR-30c-2-3p | (pC)UgGgAgAaGgCuGuUuAcUcU | 24 |
| miR30c-B5 | B5 | miR-30c-1-3p | CuGgGaGaGgGuUgUuUa(pC)U(pC)(pC)u | 25 |
| miR30c-B6 | B6 | miR-30c-1-3p | (pC)UgGgAgAgGgUuGuUuA(pC)U | 26 |
| | | | (pC)(pC)u | |
| miR30c-B7 | B7 | miR-30c-2-3p | CuGgGaGaAgG(pC)uGuUuA(pC)u(pC)U | 27 |
| miR30c-B8 | B8 | miR-30c-2-3p | (pC)UgGgAgAaGg(pC)UgUuUa | 28 |
| | | | (pC)U(pC)u | |
| miR30c-B9 | B9 | miR-30c-1-3p | (pC)(pC)(pC)UgGgAgAgGgUuGuUuAcUcC | 29 |
| miR30c-B10 | B10 | miR-30c-2-3p | (pC)(pC)(pC)UgGgAgAaGgCuGuUuAcUcU | 30 |
| miR30c-B11 | B11 | miR-30c-2-3p | (pC)(pC)(pC)ugggagaaggcuguuuacucu | 31 |

TABLE 3
MIR-30c analogs and sequences containing GalNAc and different
phosphorothioate linkages. Upper case letters, 2′-deoxy-2′-fluoro (2′-F)
ribosugar modifications; lower case letters, 2′-O-methyl (2′-OMe) ribosugar
modifications; (pC), 2′-GalNAc clicked cytidine; “•” symbol,
phosphorothioate linkages.

| | Short | | | SEQ ID |
|---|---|---|---|---|
| Compound | form | Strand | Sequence (5′ to 3′) | NO: |
| miR30c-C1 | C1 | miR-30c-1-3p | (pC)•U•gGgAgAgGgUuGuUuAcUc•C | 1 |
| miR30c-C2 | C2 | miR-30c-1-3p | (pC)•UgGgAgAgGgUuGuUuAc•U•c•C | 2 |
| miR30c-C3 | C3 | miR-30c-1-3p | (pc)•U•gGgAgAgGgUuGuUuAc•U•c•C | 3 |
| miR30c-C4 | C4 | miR-30c-1-3p | GgGaGaGgGuUgUuUaCuC(pC)•u | 4 |
| miR30c-C5 | C5 | miR-30c-1-3p | GgGaGaGgGuUgUuUaCu•C•(pC)•u | 5 |
| miR30c-C6 | C6 | miR-30c-1-3p | C•u•GgGaGaGgGuUgUuUaCuC(pC)u | 6 |

What is claimed is:

1. A double stranded polynucleotide complex, the double stranded polynucleotide complex comprising a first strand which optionally comprises no nucleotide modifications and a second strand comprising nucleotide modifications forms the double stranded polynucleotide complex with the first strand, wherein the second strand comprising the modifications comprises a sequence selected from the group sequences consisting of:

| | | | | SEQ ID |
|---|---|---|---|---|
| | | | | NO: |
| miR30c-C1 | C1 | miR-30c-1-3p | (pC)•U•gGgAgAgGgUuGuUuAcUc•C | 1 |
| miR30c-C2 | C2 | miR-30c-1-3p | (pC)•UgGgAgAgGgUuGuUuAc•U•c•C | 2 |
| miR30c-C3 | C3 | miR-30c-1-3p | (pc)•U•gGgAgAgGgUuGuUuAc•U•c•C | 3 |
| miR30c-C4 | C4 | miR-30c-1-3p | GgGaGaGgGuUgUuUaCuC(pC)•u | 4 |
| miR30c-C5 | C5 | miR-30c-1-3p | GgGaGaGgGuUgUuUaCu•C•(pC)•u | 5 |
| miR30c-C6 | C6 | miR-30c-1-3p | C•u•GgGaGaGgGuUgUuUaCuC(pC)u | 6 |

wherein upper case letters signify a 2′-deoxy-2′-fluoro (2′-F) ribosugar modification; lower case letters signify a 2′-O-methyl (2′-OMe) ribosugar modification; pC signifies 2′-a GalNAc clicked cytidine, and the symbol signifies a phosphorothioate linkage.

2. The double stranded polynucleotide complex of claim 1, wherein the second strand comprises the C2 strand.

3. The double stranded polynucleotide complex of claim 2, wherein the first strand comprises no nucleotide modifications.

4. A composition comprising the double stranded polynucleotide complex of claim 1, wherein the composition is liposome and viral vector free.

5. The composition of claim 4, wherein the second strand comprises the C2 strand.

6. The composition of claim 5, wherein the first strand comprises no nucleotide modifications.

7. A method for inhibiting apoB secretion from cells, the method comprising introducing into the cells the double stranded polynucleotide complex of claim 1.

8. The method of claim 7, wherein the second strand comprises the C2 strand.

9. The method of claim 8, wherein the first strand comprises no nucleotide modifications.

10. The method of claim 7, wherein the introducing into the cells does not reduce apoA1 secretion, or reduces apoA1 secretion less than a control value obtained from introducing into the cells a double stranded polynucleotide complex that does comprise the C2 strand.

11. The method of claim 8, wherein the introducing into the cells does not reduce apoA1 secretion, or reduces apoA1 secretion less than a control value obtained from introducing into the cells a double stranded polynucleotide complex that does comprise the C2 strand.

12. The method of claim 9, wherein the introducing into the cells does not reduce apoA1 secretion, or reduces apoA1 secretion less than a control value obtained from introducing into the cells a double stranded polynucleotide complex that does comprise the C2 strand.

13. The method of claim 7, wherein the cells are human liver cells.

14. The method of claim 8, wherein the cells are human liver cells.

15. The method of claim 9, wherein the cells are human liver cells.

16. The method of claim 10, wherein the cells are human liver cells.

17. The method of claim 11, wherein the cells are human liver cells.

18. The method of claim 12, wherein the cells are human liver cells.

19. The method of claim 8, wherein the cells are human liver cells that are present in an individual.

20. The method of claim 9, wherein the cells are human liver cells that are present in an individual.
